(19) United States
(12) Patent Application Publication
(10) Pub. No.: US 2025/0302602 A1
(43) Pub. Date: Oct. 2, 2025

(54) Artificial Sphincter

(71) Applicant: Biomagtec, Inc., Dover, DE (US)

(72) Inventors: Pourya SHOKRI, Newton, MA (US); Shahin Tabatabaei, Newton, MA (US)

(21) Appl. No.: 19/238,363

(22) Filed: Jun. 14, 2025

Related U.S. Application Data
(63) Continuation-in-part of application No. 17/181,108, filed on Feb. 22, 2021, now Pat. No. 12,329,623.
(60) Provisional application No. 62/980,155, filed on Feb. 22, 2020.

(51) Int. Cl. A61F 2/00; A61F 2/48

(52) U.S. Cl. CPC A61F 2/0036; A61F 2/482

(57) ABSTRACT

An implantable device for adjusting fluid flow through a bodily lumen, comprises a cuff configured to be positioned around the lumen; an actuator; a connection part connecting the cuff and the actuator; and an induction coil configured to generate an induced current in response to a transcutaneous magnetic induction, wherein the actuator is configured to operate the cuff by transitioning the cuff between a deployed state and an undeployed state in response to the induced current. In some embodiments, the device further comprises an alternative actuation mechanism that includes a magnet assembly configured to rotate and cause the transition of the cuff between the deployed state and the undeployed state. Further, in some embodiments, the transcutaneous magnetic induction is generated by an external inducer configured to induce a temporally varying magnetic flux in the coil to generate the induced current.

### RELATED APPLICATIONS

This application is a continuation in part of U.S. patent application Ser. No. 17/181,108, filed Feb. 22, 2021, to be issued as Patent No. 12,329,623 on Jun. 17, 2025, which claims the benefit of priority of U.S. Provisional Application No. 62/980,155 filed on Feb. 22, 2020, both titled “Artificial Urethral Sphincter”, the entire disclosures of both are incorporated herein by reference.

### TECHNICAL FIELD

The present disclosure generally relates to medical implants, and particularly relates to an artificial sphincter and, more particularly, to an artificial urethral sphincter.

### BACKGROUND

Urinary incontinence is generally defined as the involuntary leakage of urine. In simple terms, urinary incontinence is to urinate when not intended to. In other words, urinary incontinence is the inability to hold urine in the bladder because voluntary control over the urinary sphincter is either lost or weakened. Urinary inconsistence is a much more common problem than most people think. For example, the US Department of Health and Human Services estimates that approximately 13 million Americans suffer from urinary incontinence. Similarly, in the United Kingdom, at least three million people, that is, approximately 5% of the total population, are estimated to suffer from urinary incontinence.

Treatment for urinary incontinence depends on the type of incontinence, the severity of the problem, and also the underlying cause. In most cases, physicians suggest patients to try the least invasive treatments such as behavioral techniques and physical therapy, and then move on to other options only if these techniques fail. Behavioral techniques include bladder training, scheduled toilet trips, and diet management. Physical therapies include pelvic floor muscle exercises and electrical stimulation. Often, medications are used in conjunction with behavioral techniques. Such techniques, therapies, or medications, however, are not always effective or convenient.

In addition, and as alternative solutions, medical devices or more intrusive procedures may be employed. The medical devices include urethral inserts, which are small tampon-like disposable devices inserted into the urethra and act as a plug to prevent leakage.

Intrusive procedures, on the other hand, may include surgical procedures aimed at fixing problems that cause urinary incontinence. These surgical procedures include sling procedures, bladder neck suspension, and artificial urinary sphincter prostheses.

There is, therefore, a need for an artificial urinary sphincter which is simple, inexpensive, and ergonomic with minimum discomfort of the patient and minimum risk of malfunction in long-term use.

### SUMMARY

Some embodiments relate to an implantable device for adjusting fluid flow through a bodily lumen, the implantable device including: a cuff configured to be positioned around the lumen; an actuator; a connection part connecting the cuff and the actuator; and an induction coil configured to generate an induced current in response to a transcutaneous magnetic induction, wherein: the actuator is configured to operate the cuff by transitioning the cuff between a deployed state and an undeployed state in response to the induced current.

Some embodiments relate to a device, further including an alternative actuation mechanism that includes a magnet assembly configured to rotate and cause the transition of the cuff between the deployed state and the undeployed state.

Some embodiments relate to a device, wherein the lumen is a urethra, an artery, a vein, or a colon.

Some embodiments relate to a device, wherein the actuator includes a motor configured to be powered by the induced current.

Some embodiments relate to a device, wherein the motor is configured to be powered by a rectified current generated from the induced current.

Some embodiments relate to a device, wherein the actuator is configured to operate the cuff without using a battery.

Some embodiments relate to a device, wherein the actuator is configured to operate the cuff based on information derived from the induced current.

Some embodiments relate to a device, wherein the transcutaneous magnetic induction is generated by an external inducer.

Some embodiments relate to a device, wherein the external inducer is configured to induce a temporally varying magnetic flux in the coil to generate the induced current.

Some embodiments relate to a device, wherein the external inducer is configured such that a movement of the external inducer relative to the induction coils generates a varying magnetic flux in the induction coil and generating the induced current.

Some embodiments relate to an implantable device for adjusting fluid flow through a bodily lumen, the implantable device including: a cuff configured to be positioned around the lumen; an actuator; a connection part connecting the cuff and the actuator; and a fail-safe mechanism, wherein: the actuator is configured to operate the cuff by transitioning the cuff between a deployed state and an undeployed state; and the fail-safe mechanism is configured to transition the cuff from the deployed state to the undeployed state.

Some embodiments relate to a device, wherein the fail-safe mechanism includes a magnet assembly configured to rotate and cause the transition of the cuff from the deployed state to the undeployed state.

Some embodiments relate to a device, wherein the fail-safe mechanism includes a release mechanism configured to disengage the actuator from the cuff.

Some embodiments relate to a device, wherein the fail-safe mechanism is configured to be operated when the actuator fails to operate the cuff by transitioning the cuff from the deployed state to the undeployed state.

Some embodiments relate to an implantable device for controllably covering a body part, the device including: an actuator; and a belt including: a cuff configured to wrap around at least part of the body part; a connection part connecting the cuff and the actuator, wherein: the cuff has two ends configured to be fixedly connected to position the cuff around the body part; the actuator is configured to operate the belt by transitioning the belt between a deployed state and an undeployed state; and the cuff provides an intervening sheath between the connection part and the body part.

Some embodiments relate to a device, wherein the cuff includes a mesh.

Some embodiments relate to a device, wherein in the deployed state the cuff buckles around the body part to increase a pressure on the body part.

Further understanding of various aspects of the embodiments may be obtained by reference to the following detailed description in conjunction with the associated drawings, which are described briefly below.

### BRIEF DESCRIPTION OF THE DRAWINGS

The drawings are not necessarily to scale or exhaustive. Instead, emphasis is generally placed upon illustrating the principles of the embodiments described herein. The accompanying drawings, which are incorporated in this specification and constitute a part of it, illustrate several embodiments consistent with the disclosure. Together with the description, the drawings serve to explain the principles of the disclosure.

In the drawings:

FIG. 1A illustrates an exemplary artificial urethral sphincter, consistent with one or more exemplary embodiments of the present disclosure.

FIG. 1B illustrates a cuff member, consistent with one or more exemplary embodiments of the present disclosure.

FIG. 1C illustrates an artificial urethral sphincter when the artificial urethral sphincter is implanted inside a patient's body, consistent with one or more exemplary embodiments of the present disclosure.

FIG. 1D illustrates an open view of a cuff member, consistent with one or more exemplary embodiments of the present disclosure.

FIG. 2A illustrates an artificial urethral sphincter, consistent with one or more exemplary embodiments of the present disclosure.

FIG. 2B illustrates an artificial urethral sphincter, consistent with one or more exemplary embodiments of the present disclosure.

FIG. 2C illustrates an artificial urethral sphincter in a scenario in which a cuff member grips a urethra of a patient and the urethra of the patient is blocked, consistent with one or more exemplary embodiments of the present disclosure.

FIG. 2D illustrates an artificial urethral sphincter in a scenario in which a urethra of a patient is released and unblocked, consistent with one or more exemplary embodiments of the present disclosure.

FIG. 2E illustrates an artificial urethral sphincter when the artificial urethral sphincter is implanted inside a patient's body, consistent with one or more exemplary embodiments of the present disclosure.

FIG. 3 illustrates an artificial urethral sphincter, consistent with one or more exemplary embodiments of the present disclosure.

FIG. 4A illustrates an artificial urethral sphincter, consistent with one or more exemplary embodiments of the present disclosure.

FIG. 4B illustrates a perspective view of an adjustment cylinder, consistent with one or more exemplary embodiments of the present disclosure.

FIG. 4C illustrates a perspective view of a hollow cylinder, consistent with one or more exemplary embodiments of the present disclosure.

FIG. 4D illustrates a cap part, consistent with one or more exemplary embodiments of the present disclosure.

FIG. 5A illustrates a bottom perspective view of an artificial urethral sphincter, consistent with one or more exemplary embodiments of the present disclosure.

FIG. 5B illustrates a side view of an artificial urethral sphincter, consistent with one or more exemplary embodiments of the present disclosure.

FIG. 5C illustrates an exploded view of an artificial urethral sphincter, consistent with one or more exemplary embodiments of the present disclosure.

FIG. 6A illustrates an artificial urethral sphincter, consistent with one or more exemplary embodiments of the resent disclosure.

FIG. 6B illustrates an artificial urethral sphincter, consistent with one or more exemplary embodiments of the resent disclosure.

FIG. 6C illustrates an artificial urethral sphincter, consistent with one or more exemplary embodiments of the resent disclosure.

FIG. 6D illustrates an artificial urethral sphincter, consistent with one or more exemplary embodiments of the resent disclosure.

FIG. 6E illustrates an artificial urethral sphincter, consistent with one or more exemplary embodiments of the resent disclosure.

FIG. 6F illustrates an artificial urethral sphincter in a scenario in which a second end of a spring is pulled toward a first end of a spring, consistent with one or more exemplary embodiments of the present disclosure.

FIG. 6G illustrates an artificial urethral sphincter in a scenario in which a second end of a spring is pulled toward a first end of a spring, consistent with one or more exemplary embodiments of the present disclosure.

FIG. 7 is a diagram illustrating an artificial urethral sphincter according to some embodiments.

FIG. 8A is a diagram illustrating a cuff and a connection part according to some embodiments.

FIG. 8B is a diagram illustrating an example of an implementation of the cuff and the connection part of FIG. 8A in which the cuff encircles a urethra according to some embodiments.

FIG. 8C shows some more details of cable jacket according to some embodiments.

FIG. 9A is a diagram showing an outer structure of an actuator according to some embodiments.

FIGS. 9B-9G are diagrams showing some details of the actuator of FIG. 9A according to some embodiments.

FIGS. 9H and 9I are diagrams showing some details of an emergency button of the actuator of FIG. 9A according to some embodiments.

FIG. 9J is a diagram illustrating functionality of a second pulley of the actuator of FIG. 9A according to some embodiments.

FIGS. 9K and 9L are diagrams illustrating functionality of the emergency button according to some embodiments.

FIG. 9M is a schematic diagram showing an example of an implementation in which the actuator of FIG. 9A is operated by an electronic device according to some embodiments.

FIG. 9N schematically illustrates an example of an embodiment in which the actuator is operated by a magnet M.

FIGS. 10A and 10B are diagrams illustrating a cover assembly according to some embodiments.

FIG. 11 is a diagram illustrating a complex including a motor, a magnet assembly, a gear assembly, and a first pulley according to some embodiments.

FIG. 12 is a schematic diagram depicting an example of an implementation of a controller according to some embodiments.

FIGS. 13A-13C show some details of a cuff and a connection part according to some embodiments.

FIGS. 13D-13K illustrate various features of cuff enabling the cuff to tighten or loosen around a body lumen according to various embodiments.

FIGS. 14A-14C show some details of a controllable cover including a cuff and a connection part according to some embodiments.

FIGS. 14D and 14E illustrate cuff, connection part, and bladder when cuff is in a deployed or buckled state, according to some embodiments.

### DETAILED DESCRIPTION

The following detailed description refers to the accompanying drawings. The same or similar reference numbers may have been used in the drawings or in the description to refer to the same or similar parts. Also, similarly named elements may perform similar functions and may be similarly designed, unless specified otherwise. Details are set forth to provide an understanding of the exemplary embodiments. Embodiments, e.g., alternative embodiments, may be practiced without some of these details. In other instances, well known techniques, procedures, and components have not been described in detail to avoid obscuring the described embodiments.

Herein is disclosed an artificial urethral sphincter. An exemplary artificial urethral sphincter may include a cuff member, a hollow cylinder, a spring, and a cable. The cuff member may be wrapped around a urethra of a patient and a distal end of the cuff member may be attached to a proximal end of the cuff member. A first part of the cable may be disposed inside an internal pocket of the cuff member. A first end of the cable, which may be connected to the first part of the cable, may be attached to the distal end of the cuff member. The second part of the cable may be disposed inside the hollow cylinder. The spring may be disposed inside the hollow cylinder. A second end of the cable, which may be connected to the second part of the cable, may be attached to a top end of the spring.

In an exemplary embodiment, the spring may push up the second end of the cable inside the hollow cylinder and, thereby, pulling out the first part of the cable from the internal pocket and blocking the urethra of the patient. A magnetic part may be attached to the top end of the spring. In an exemplary scenario, when a user intends to urinate, the user may pull down a first end of an exemplary spring by moving a magnet toward a bottom end of the spring. When a user intends to urinate, the user may move a magnet close to the bottom end of the spring in such a way that a distance between the magnet and the bottom end of the spring becomes less than 1 centimeter. By pulling down the first end of the spring, the cable may be loosened and the urethra of the patient may be unblocked. By utilizing such an artificial urethral sphincter, when a urethra of a patient is blocked by the cuff member and an undesired excess force is applied to the bladder of the patient, the applied excess force may urge the cuff member to unblock the urethra of the patient and, to thereby, may prevent any negative consequences of urinary retention in the bladder of the patient. In other words, the disclosed artificial urethral sphincter artificial urethral sphincter may provide a safety facility for a patient.

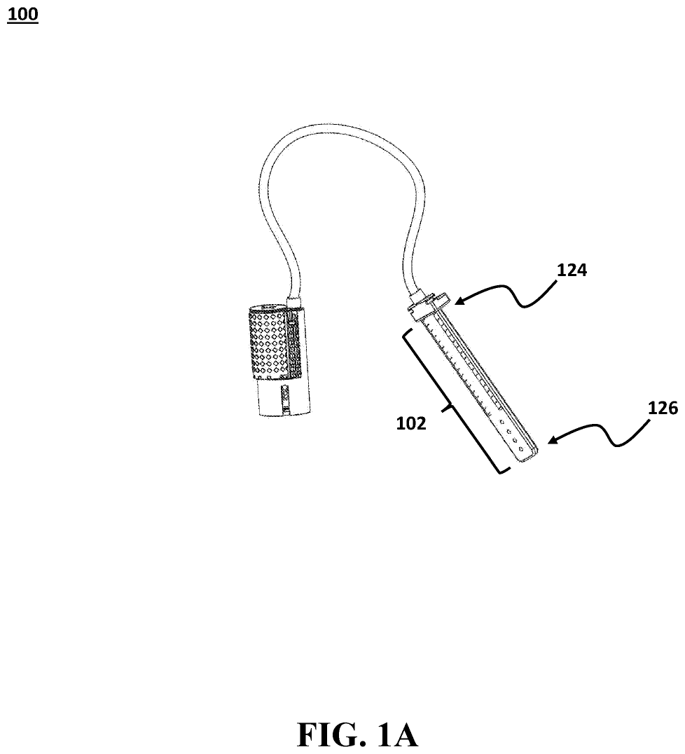

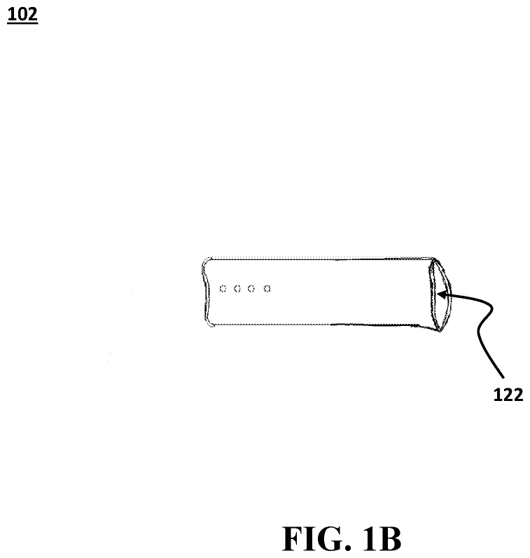

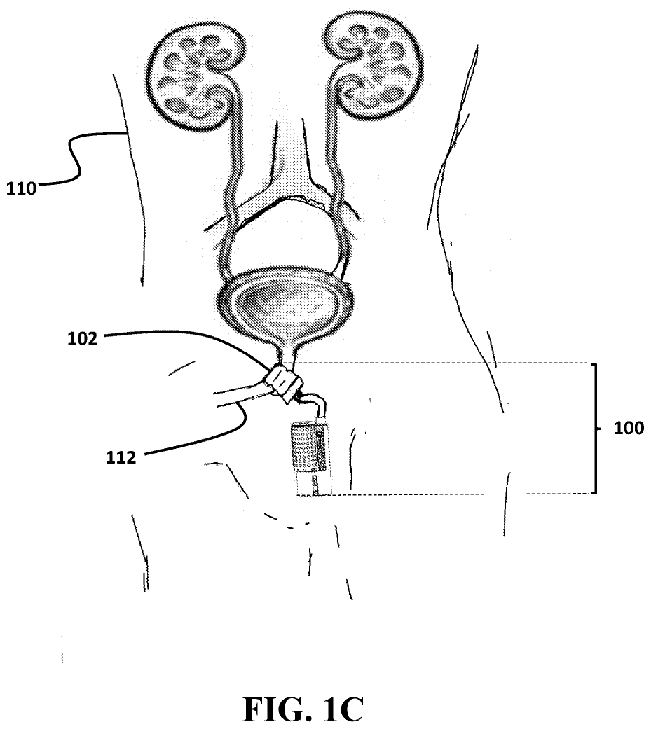

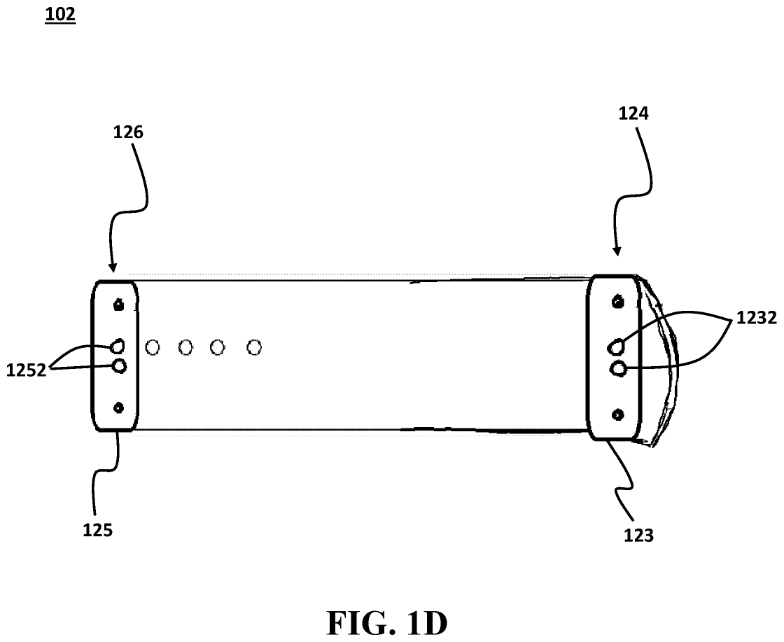

FIG. 1A shows an exemplary artificial urethral sphincter 100, consistent with one or more exemplary embodiments of the present disclosure. As shown in FIG. 1A, artificial urethral sphincter 100 may include a cuff member 102. FIG. 1B shows cuff member 102, consistent with one or more exemplary embodiments of the present disclosure. As shown in FIG. 1B, cuff member 102 may include an internal pocket 122.

FIG. 1C shows artificial urethral sphincter 100 implanted inside a patient's 110 body, consistent with one or more exemplary embodiments of the present disclosure. As shown in FIG. 1C, cuff member 102 may be configured to encircle a urethra 112 of patient 110. Patient 110 may refer to a person who has a urethra, and is unable to hold urine in the bladder because voluntary control over the urinary sphincter is either lost or weakened. When cuff member 102 encircle urethra 112 of patient 110, cuff member 102 may form a circle around urethra 112 of patient 110. Cuff member 102 may be made up of a flexible material which may allow cuff member 102 to move or deform easily. A user may wrap cuff member 102 around urethra 112 of patient 110. The user may refer to a surgeon. Cuff member 102 may act as a belt around urethra 112 of patient 110. A user may then attach a proximal end 124 of cuff member 102 to a distal end 126 of cuff member 102. FIG. 1D shows an open view of cuff member 102, consistent with one or more exemplary embodiments of the present disclosure.

As shown in FIG. 1D, cuff member 102 may include a first attaching member 123 at proximal end 124 of cuff member 102. Cuff member 102 may further include a second attaching member 125 at distal end 126 of cuff member 102. A user, after wrapping cuff member 102 around urethra 112 of patient 110, may attach first attaching member 123 to second attaching member 125. First attaching member 123 may include a first pair of suture holes 1232. Second attaching member 125 may include a second pair of suture holes 1252. A user, after wrapping cuff member 102 around urethra 112 of patient 110, may attach first attaching member 123 to second attaching member 125 through suturing first attaching member 123 to second attaching member 125 by utilizing first pair of suture holes 1232 and second pair of suture holes 1252.

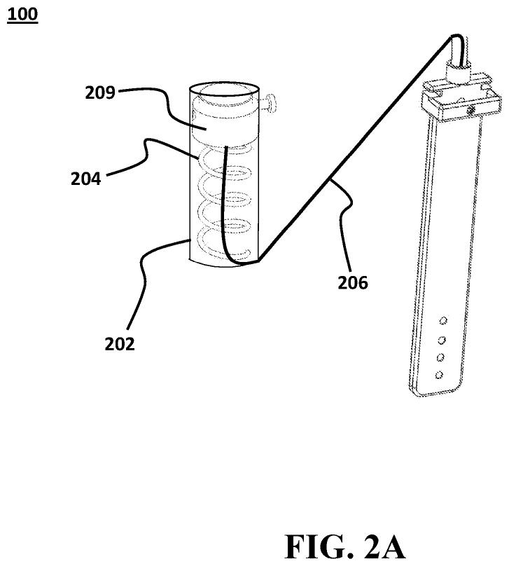

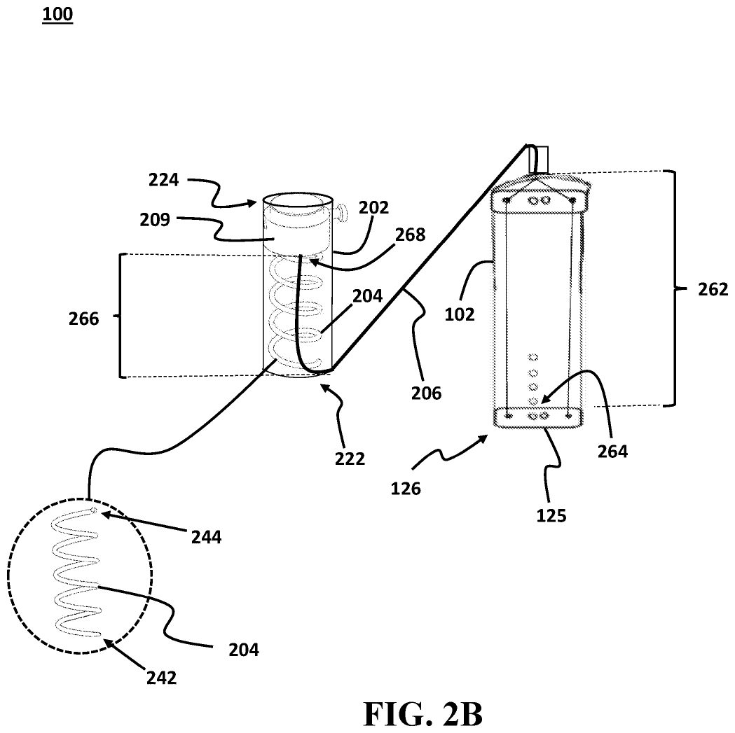

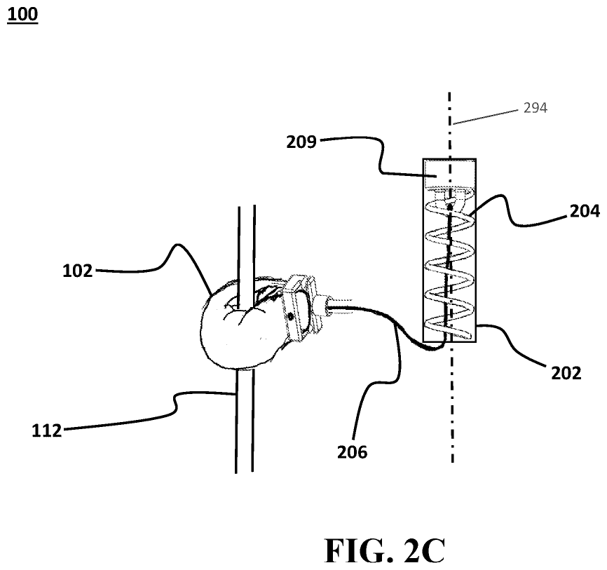

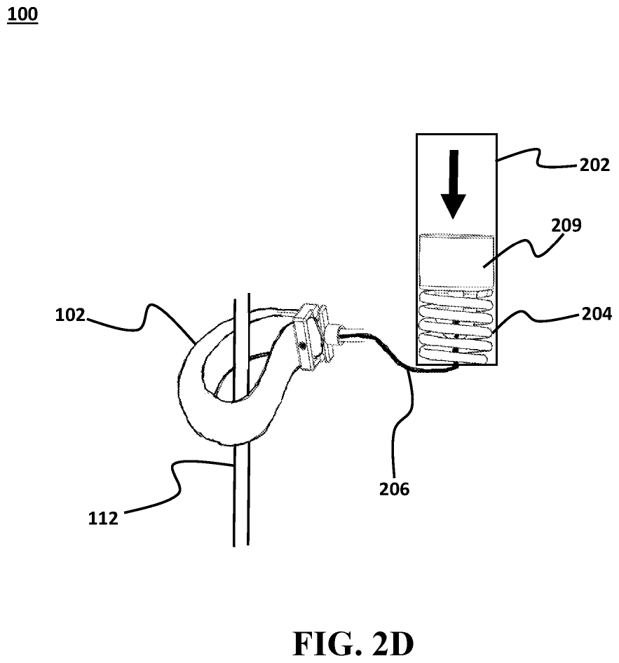

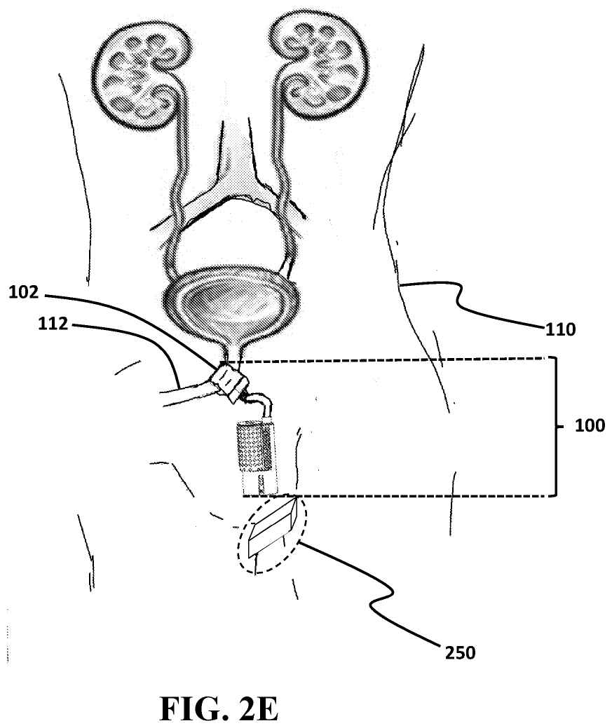

FIG. 2A shows artificial urethral sphincter 100, consistent with one or more exemplary embodiments of the present disclosure. As shown in FIG. 2A, artificial urethral sphincter 100 may further include a hollow cylinder 202. Hollow cylinder 202 may be disposed inside patient's 110 body. A user may dispose hollow cylinder 202 inside a perineum (not illustrated) of patient 110. Artificial urethral sphincter 100 may further include a spring 204. Spring 204 may be disposed inside hollow cylinder 202. Spring 204 may be replaced with any elastic object that stores mechanical energy and a length of the object changes when an external force is applied to the object. For example, spring 204 may be replaced with an elastic rubber.

FIG. 2B shows artificial urethral sphincter 100, consistent with one or more exemplary embodiments of the present disclosure. As shown in FIG. 2A and FIG. 2B, artificial urethral sphincter 100 may further include a cable 206. A first part 262 of cable 206 may be disposed inside internal pocket 122. A first end 264 of cable 206 may be attached to distal end 126 of cuff member 102. First end 264 of cable 206 may be attached to second attaching member 125. A second part 266 of cable 206 may be disposed inside hollow cylinder 202. Second part 266 of cable 206 may be disposed inside spring 204. Second part 266 of cable 206 may be disposed outside spring 204 and inside hollow cylinder 202. Second end 268 of cable 206 may be attached to a second end 244 of spring 204. Spring 204 may push second end 244 of spring 204 and second end 268 of cable 206 toward a second end 224 of hollow cylinder 202. When spring 204 pushes second end 244 of spring 204 and second end 268 of cable 206 toward second end 224 of hollow cylinder 202, first part 262 of cable 206 may be pulled out of internal pocket 122. When first part 262 of cable 206 is pulled out from internal pocket 122, cuff member 102 may shrink and, thereby, cuff member 102 may grip urethra 112 of patient 110. When cuff member 102 grips urethra 112 of patient 110, urethra 112 of patient 110 may be blocked and, consequently, urine may not be allowed to pass through urethra 112 of patient 110.

FIG. 2C shows artificial urethral sphincter 100 in a scenario in which cuff member 102 grips urethra 112 of patient 110 and urethra 112 of patient 110 is blocked, consistent with one or more exemplary embodiments of the present disclosure.

Artificial urethral sphincter 100 may further include a moveable part 209. Moveable part 209 may be disposed slidably inside hollow cylinder 202. Moveable part 209 may include a magnet. Moveable part 209 may be made up of a magnetic material. When moveable part 209 is disposed slidably inside hollow cylinder 202, it may mean that moveable part 209 is disposed inside hollow cylinder 202 in such a way that moveable part 209 is allowed to move linearly inside hollow cylinder 202. Moveable part 209 may be allowed to move linearly along a slide axis 294 inside hollow cylinder 202. Slide axis 294 may coincide a main longitudinal axis of hollow cylinder 202. Moveable part 209 may be attached to second end 244 of spring 204. Moveable part 209 may be disposed onto spring 204. Second end 268 of cable 206 may be attached to a second end 244 of spring 204 through attaching second end 268 of cable 206 to moveable part 209. When moveable part 209 moves toward a first end 222 of hollow cylinder 202, cable 206 may become loose. A cable may become loose when a tensile stress in the cable is zero. In other words, a cable may be loose when no external tensile force is applied to the cable. When cable 206 becomes loose, first part 262 of cable 206 may be loosened accordingly and, thereby, cuff member 102 may release urethra 112 of patient 110. When urethra 112 of patient 110 is released, urethra 112 of patient 110 may be unblocked. When urethra 112 of patient 110 is unblocked, urine may be discharged from bladder and through urethra 112 of patient 110. FIG. 2D shows artificial urethral sphincter 100 in a scenario in which urethra 112 of patient 110 is released and unblocked, consistent with one or more exemplary embodiments of the present disclosure.

Moveable part 209 may be made up of a magnetic material. When a part of a magnetic material is disposed inside a magnetic field of a magnet, the part may be urged to move toward the magnet. When a magnet is disposed near to first end 222 of hollow cylinder 202, moveable part 209 may be attracted toward first end 222 of hollow cylinder 202. Patient 110 may unblock urethra 112 of patient 110 by moving a magnet toward first end 222 of hollow cylinder 202. Patient 110 may unblock urethra 112 of patient 110 by moving a magnet close to first end 222 of hollow cylinder 202 in such a way that a distance between the magnet and first end 222 of hollow cylinder 202 becomes less than 1 centimeter. In an absence of an external magnetic field, moveable part 209 may be placed at second end 224 of hollow cylinder 202 and, consequently, urethra 112 of patient 110 may be blocked as discussed above. Then, due to an absence of an external magnetic field, urine may not be allowed to pass through urethra 112 of patient 110. When patient 110 intends to urinate, patient 110 may allow urine discharge from patient's 110 bladder and through urethra 112 of patient 110 by disposing a magnet near to second end 224 of hollow cylinder 202. Cylinder 202 may be disposed inside patient's 110 body in such a way that second end 224 of hollow cylinder 202 is located near to patient's 110 skin so that patient 110 may be able to easily dispose a magnet near to second end 224 of hollow cylinder 202. FIG. 2E shows artificial urethral sphincter 100 when artificial urethral sphincter 100 is implanted inside a patient's 110 body, consistent with one or more exemplary embodiments of the present disclosure. As shown in FIG. 2E, patient 110 may dispose a magnet 250 near to second end 224 of hollow cylinder 202 to unblock urethra 112 of patient 110 and then urine may be discharged from patient's 110 bladder. Magnet 250 may include a magnetic part, a magnetic inductor, an electromagnetic inductor, or a combination thereof.

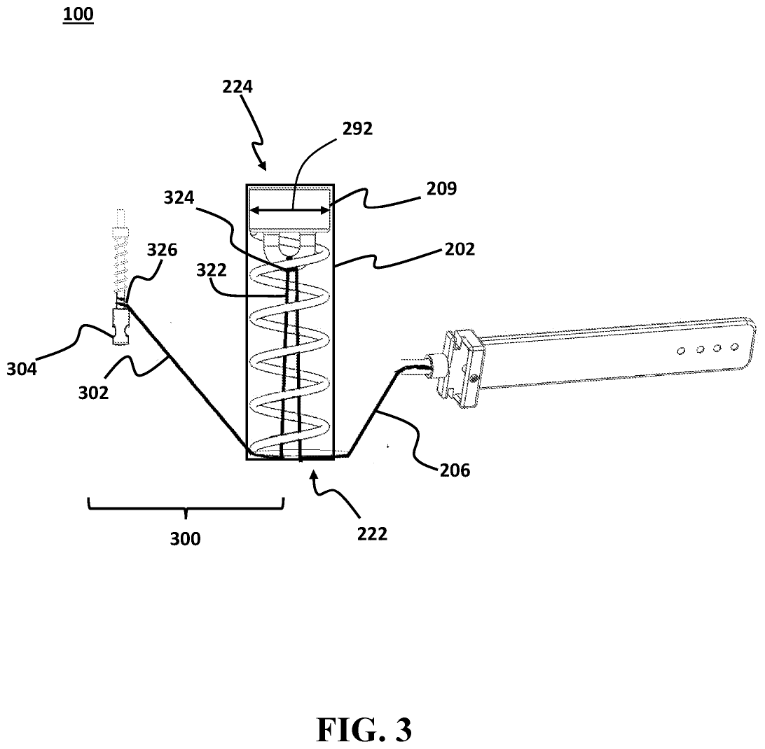

FIG. 3 shows artificial urethral sphincter 100, consistent with one or more exemplary embodiments of the present disclosure. As shown in FIG. 3, artificial urethral sphincter 100 may further include a first cuff adjustment mechanism 300. First cuff adjustment mechanism 300 may include an adjustment cable 302. A first part 322 of adjustment cable 302 may be disposed inside hollow cylinder 202. A first end 324 of adjustment cable 302 may be attached to second end 268 of cable 206. First end 324 of adjustment cable 302 may be attached to second end 268 of cable 206 in such a way that adjustment cable 302 and cable 206 create a unitary/integrated cable.

First end 324 of adjustment cable 302 may be attached to second end 244 of spring 204 through attaching first end 324 of adjustment cable 302 to moveable part 209. First part 322 of adjustment cable 302 may be disposed inside spring 204. Spring 204 may be disposed inside hollow cylinder 202.

First cuff adjustment mechanism 300 may further include an adjustment screw 304. A second end 326 of adjustment cable 302 may be attached to adjustment screw 304. A second part of adjustment cable 302 may be wrapped around adjustment screw 304. When adjustment screw 304 is twisted in a first direction, more length of adjustment cable 302 may be wrapped around adjustment screw 304 and, consequently, moveable part 209 may move toward first end 222 of hollow cylinder 202. When moveable part 209 moves toward first end 222 of hollow cylinder 202, cable 206 may be loosened and, consequently, a gripping force that may be applied from cuff member 102 to urethra 112 of patient 110 may decrease. The gripping force may refer to a normal force that may be applied from cuff member 102 to urethra 112 of patient 110 in order to block urethra 112 of patient 110. When adjustment screw 304 is twisted in a second direction, less length of adjustment cable 302 may be maintained wrapped around adjustment screw 304 and, consequently, moveable part 209 may move toward second end 224 of hollow cylinder 202. When moveable part 209 moves toward second end 224 of hollow cylinder 202, cable 206 may be tightened and, consequently, the gripping force that may be applied from cuff member 102 to urethra 112 of patient 110 may increase.

First cuff adjustment mechanism 300 may be used for tightening cable 206. After that artificial urethral sphincter 100 is implanted, first cuff adjustment mechanism 300 may be used to tighten cable 206 so that cable 206 is able to transfer the gripping force appropriately. First cuff adjustment mechanism 300 may allow a same size artificial urethral sphincter 100 to be used for different patients with different urethra sizes. When moveable part 209 is moved down inside hollow cylinder 202, cable 206 may be loosened. First cuff adjustment mechanism 300 may be used to tighten cable 206 and compensate the looseness of cable 206.

First cuff adjustment mechanism 300 may provide significant benefits. For example, a user, for example the surgeon or patient 110 may be able to control the gripping force applied from cuff member 102 to urethra 112 of patient 110 by twisting adjustment screw 304 in the first direction and/or the second direction. For example, the surgeon may be able to increase the gripping force applied from cuff member 102 to urethra 112 of patient 110 by twisting adjustment screw 304 in a clockwise direction and decrease the gripping force applied from cuff member 102 to urethra 112 of patient 110 by twisting adjustment screw 304 in a counterclockwise direction. In instances, without utilizing first cuff adjustment mechanism 300, when magnet 250 is disposed near to second end 224 of hollow cylinder 202, moveable part 209 may be placed at second end 224 of hollow cylinder 202 and, consequently, urethra 112 of patient 110 may be gripped tightly by cuff member 102. On the other hand, in absence of magnet 250, moveable part 209 may be placed at first end 222 of hollow cylinder 202, cuff member 102 may fully release urethra 112 of patient 110 urethra 112 of patient 110. Hence, without utilizing first cuff adjustment mechanism 300, the surgeon or patient 110 may not have a full control on the gripping force applied from cuff member 102 to urethra 112 of patient 110. Adjustment screw 304 may be disposed inside patient's 110 body in such a way that adjustment screw 304 is located near to patient's 110 skin so that a surgeon or patient 110 may be able to easily twist adjustment screw 304 in clockwise or counterclockwise direction.

Extra gripping force may damage urethra 112 of patient 110, that is applying more than a threshold amount of force on urethra 112. The threshold amount of force may be enough so that cuff member 102 grips urethra 112 but does not damage it. Consequently, at first stages of using artificial urethral sphincter 100 for patient 110, adjustment screw 304 may be adjusted in such a way that cuff member 102 applies a relatively low force to urethra 112 of patient 110 so as to minimize a probable damage to urethra 112 of the patient 110. Applying a relatively low force to urethra 112 of patient 110 may refer to applying a pressure between 1000 Pascal and 2500 Pascal to urethra 112 of patient 110. But after using artificial urethral sphincter 100 for a patient for a long time, the previously applied force to urethra 112 of patient 110 may no longer be able to fully grip and block urethra 112 of patient 110. In this scenario, adjustment screw 304 may be twisted in the second direction so as to increase the gripping force applied from cuff member 102 to urethra 112 of patient 110 and, consequently, urine leakage from the urethra 112 of patient 110 may be prevented or otherwise minimized. The gripping force may refer to a normal force that may be applied from cuff member 102 to urethra 112 of patient 110 in order to block urethra 112 of patient 110. After a period of using artificial urethral sphincter 100, urethra 112 of patient 110 may be atrophied and consequently, a size of artificial urethral sphincter 100 may be changed to grip urethra 112 of patient 110 more tightly. In order to change the size of artificial urethral sphincter 100 to grip urethra 112 of patient 110 more tightly, moveable part 209 may be moved down inside hollow cylinder 202 and, thereby, cable 206 may be loosened. In order to tighten first cable 206 and compensate the looseness of cable 206, adjustment screw 304 may be twisted in the second direction so as to increase the gripping force applied from cuff member 102 to urethra 112 of patient 110.

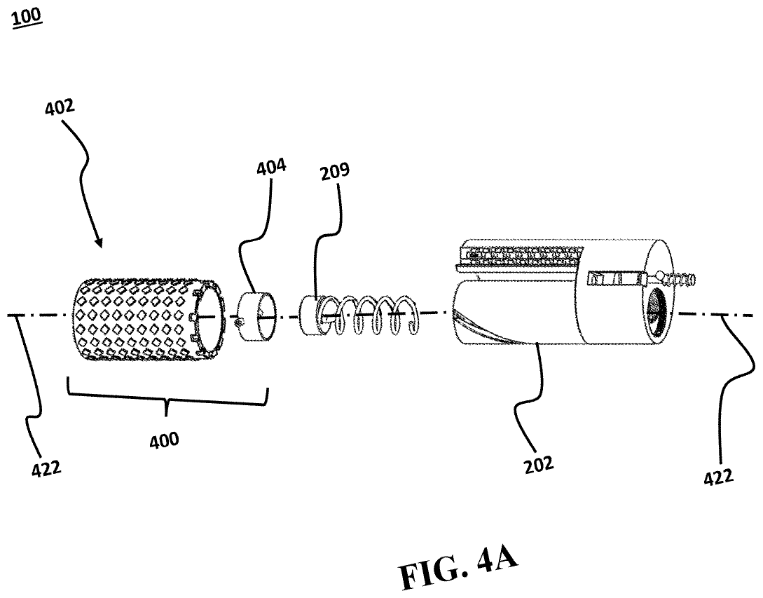

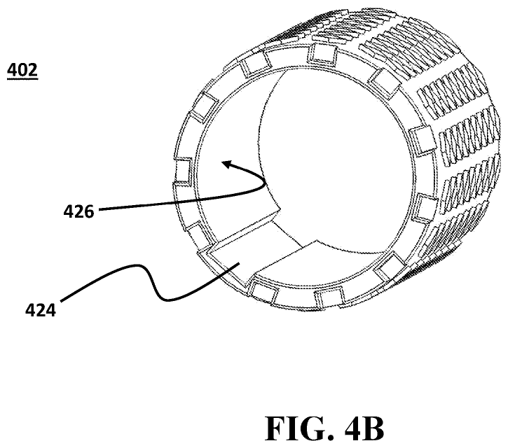

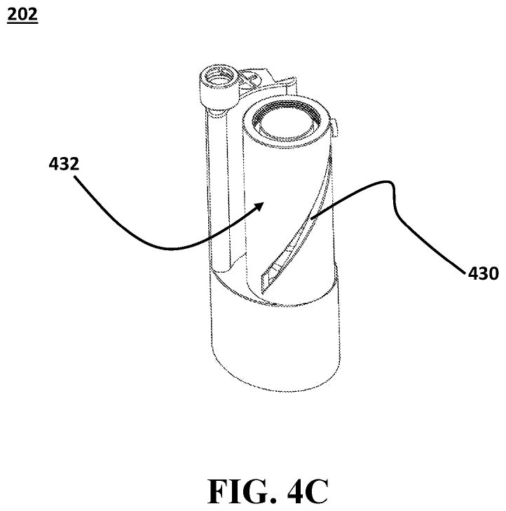

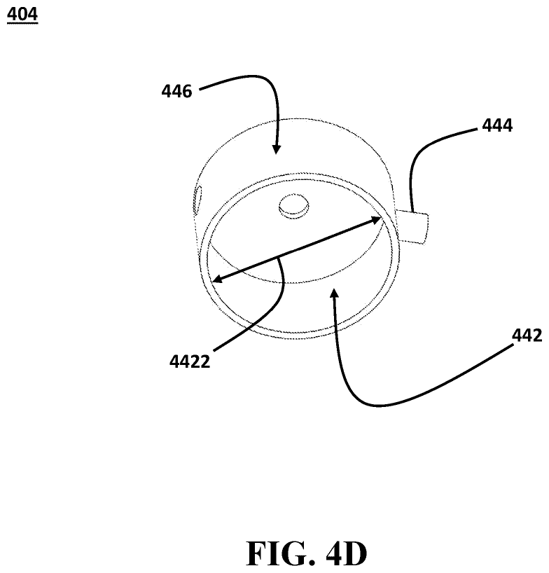

FIG. 4A shows artificial urethral sphincter 100, consistent with one or more exemplary embodiments of the present disclosure. As shown in FIG. 4A, artificial urethral sphincter 100 may include a second cuff adjustment mechanism 400. Second cuff adjustment mechanism 400 may be configured to adjust the maximum gripping force applied to urethra 112 of patient 110 from cuff member 102. Second cuff adjustment mechanism 400 may include an adjustment cylinder 402. Adjustment cylinder 402 may be disposed slidably and rotatably around hollow cylinder 202. When adjustment cylinder 402 is disposed rotatably around hollow cylinder 202, it may mean that adjustment cylinder 402 is disposed around hollow cylinder 202 in such a way that adjustment cylinder 402 is able to rotate around an axis such as a rotation axis 422. Rotation axis 422 may be the same as a main axis of hollow cylinder 202 and adjustment cylinder 402. When adjustment cylinder 402 is disposed rotatably around hollow cylinder 202, it may be the same as a scenario in which hollow cylinder 202 is disposed rotatably inside adjustment cylinder 402.

FIG. 4B shows a perspective view of adjustment cylinder 402, consistent with one or more exemplary embodiments of the present disclosure. As shown in FIG. 4B, adjustment cylinder 402 may include a longitudinal slot 424. Longitudinal slot 424 may be provided on an inner surface 426 of adjustment cylinder 402. Longitudinal slot 424 may be provided on inner surface 426 of adjustment cylinder 402 in such a way that a main axis of longitudinal slot 424 is parallel to rotation axis 422.

FIG. 4C shows a perspective view of hollow cylinder 202, consistent with one or more exemplary embodiments of the present disclosure. Hollow cylinder 202 may include a helical slot 430 on an outer surface 432 of hollow cylinder 202.

Second cuff adjustment mechanism 400 may further include a cap part 404. Cap part 404 may be disposed slidably and rotatably inside hollow cylinder 202. When cap part 404 is disposed slidably and rotatably inside hollow cylinder 202, it may mean that cap part 404 is disposed inside hollow cylinder 202 in such a way that cap part 404 is able to rotate around rotation axis 422 and move linearly along rotation axis 422. Cap part 404 may be disposed onto moveable part 209. In an exemplary scenario when cap part 404 is disposed onto moveable part 209, when cap part 404 moves downward inside hollow cylinder 202, cap part 404 may urge moveable part 209 to move downwardly with moveable part 209 but when cap part 404 moves upward inside hollow cylinder 202, moveable part 209 may not follow cap part 404.

FIG. 4D shows cap part 404, consistent with one or more exemplary embodiments of the present disclosure. As shown in FIG. 4D, cap part 404 may include an inner chamber 442. Inner chamber 442 may be configured to receive moveable part 209. A diameter 4422 of inner chamber 442 may be slightly larger than an outer diameter 292 of moveable part 209.

Cap part 404 may further include a pin 444 on an outer surface 446 of cap part 404. Pin 444 may be disposed slidably inside longitudinal slot 424 of adjustment cylinder 402. When pin 444 is disposed slidably inside longitudinal slot 424 of adjustment cylinder 402, it may mean that pin 444 may be disposed inside longitudinal slot 424 of adjustment cylinder 402 in such a way that pin 444 is able to move linearly inside longitudinal slot 424 of adjustment cylinder 402. In an exemplary scenario when pin 444 is disposed slidably inside longitudinal slot 424 of adjustment cylinder 402, when cap part 404 moves upward and downward inside hollow cylinder 202, pin 444 may also move upward and downward inside longitudinal slot 424 of adjustment cylinder 402. Moving upward inside hollow cylinder 202 may refer to a movement inside hollow cylinder 202 toward second end 224 of hollow cylinder 202. Moving downward inside hollow cylinder 202 may refer to a movement inside hollow cylinder 202 toward first end 222 of hollow cylinder 202.

Pin 444 may be disposed inside helical slot 430. When pin 444 is disposed inside helical slot 430 and cap part 404 rotates around rotation axis 422, inner surfaces of helical slot 430 may urge pin 444 to move inside helical slot 430. For example, when cap part 404 rotates around rotation axis 422 in a clockwise direction, helical slot 430 may urge pin 444 to move downwardly inside helical slot 430. Then, when cap part 404 rotates around rotation axis 422 in a clockwise direction, cap part 404 may move downwardly inside hollow cylinder 202. Also, when cap part 404 rotates around rotation axis 422 in a counterclockwise direction, helical slot 430 may urge pin 444 to move upwardly inside helical slot 430. Then, it may be understood that when cap part 404 rotates around rotation axis 422 in a counterclockwise direction, cap part 404 may move upwardly inside hollow cylinder 202.

Pin 444 may be disposed inside longitudinal slot 424 and helical slot 430. When adjustment cylinder 402 rotates around rotation axis 422 in a clockwise direction, cap part 404 may rotate around rotation axis 422 synchronously with adjustment cylinder 402 since pin 444 is disposed inside longitudinal slot 424 of adjustment cylinder 402. On the other hand, when cap part 404 rotates around rotation axis 422 in a clockwise direction, cap part 404 may move downward inside hollow cylinder 202. When adjustment cylinder 402 rotates around rotation axis 422 in a counterclockwise direction, cap part 404 may rotate around rotation axis 422 synchronously with adjustment cylinder 402 since pin 444 is disposed inside longitudinal slot 424 of adjustment cylinder 402. On the other hand, when cap part 404 rotates around rotation axis 422 in a counterclockwise direction, cap part 404 may move upward inside hollow cylinder 202.

A surgeon and/or any other user may be able to move cap part 404 downward inside hollow cylinder 202 by rotating adjustment cylinder 402 in a clockwise direction around rotation axis 422. Also, a surgeon and/or any other user may be able to move cap part 404 upward inside hollow cylinder 202 by rotating adjustment cylinder 402 in a counterclockwise direction around rotation axis 422. A surgeon and/or a user may be able to adjust the maximum gripping force that may be applied from cuff member 102 to urethra 112 of patient 110 by rotating adjustment cylinder 402 in a clockwise direction and/or a counterclockwise direction around rotation axis 422. A higher position of cap part 404 inside hollow cylinder 202 may mean that moveable part 209 may be allowed to move upper inside hollow cylinder 202 and, consequently, a greater gripping force may be applied from cuff member 102 to urethra 112 of patient 110. Hence, a user may be able to increase the maximum gripping force that may be applied from cuff member 102 to urethra 112 of patient 110 by rotating adjustment cylinder 402 in a clockwise direction around rotation axis 422.

When adjustment cylinder 402 is rotated in a clockwise direction around rotation axis 422, cap part 404 may move upper inside hollow cylinder 202 which may allow moveable part 209 to move upper inside hollow cylinder 202. When moveable part 209 is able to move upper inside hollow cylinder 202, cuff member 102 may be able to grip urethra 112 of patient 110 more tightly. Also, a surgeon may be able to decrease the maximum gripping force that may be applied from cuff member 102 to urethra 112 of patient 110 by rotating adjustment cylinder 402 in a counterclockwise direction around rotation axis 422. Adjustment cylinder 402 may be located near to patient's 110 skin so that a user may be able to easily rotate adjustment cylinder 402 around rotation axis 422.

Second cuff adjustment mechanism 400 may provide significant benefits. For example, when artificial urethral sphincter 100 is damaged and unable to function appropriately, for example due to a car accident, a surgeon may be able to easily rotate adjustment cylinder 402 around rotation axis 422 in order to unblock urethra 112 of patient 110 and, thereby, the bladder of patient 110 may be discharged.

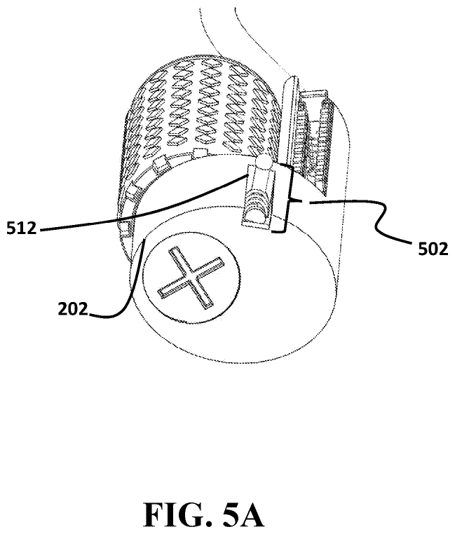

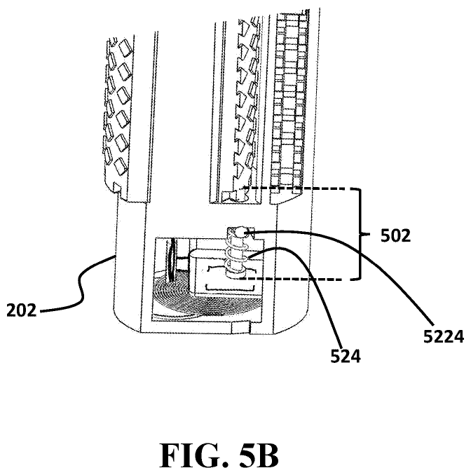

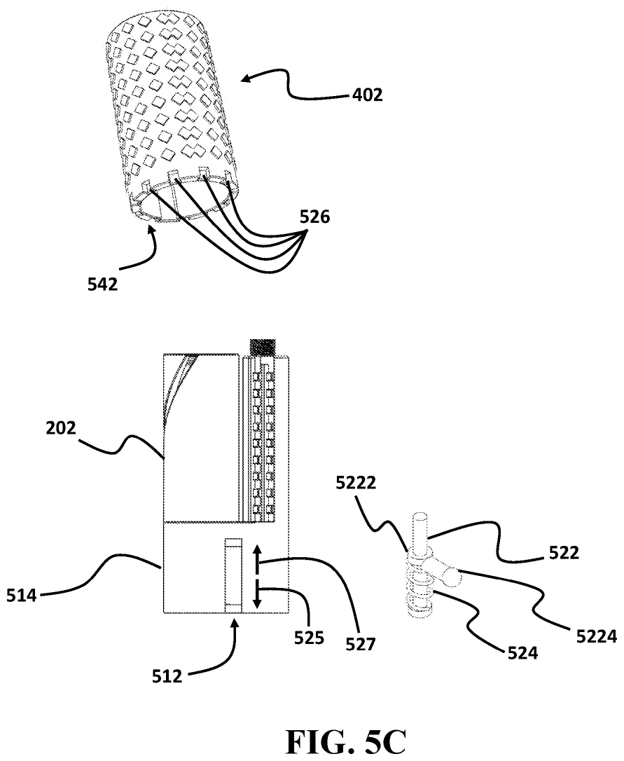

FIG. 5A shows a bottom perspective view of artificial urethral sphincter 100, consistent with one or more exemplary embodiments of the present disclosure. FIG. 5B shows a side view of artificial urethral sphincter 100, consistent with one or more exemplary embodiments of the present disclosure. As shown in FIG. 5A and FIG. 5B, second cuff adjustment mechanism 400 may include a lock mechanism 502.

FIG. 5C shows an exploded view of artificial urethral sphincter 100, consistent with one or more exemplary embodiments of the present disclosure. As shown in FIG. 5C, lock mechanism 502 may include a lock pin 522, a lock spring 524, and a plurality of pin receiving holes 526. Lock pin 522 may be disposed slidably inside a lock hole 512 of a base 514. Base 514 may be attached to hollow cylinder 202. Base 514 and hollow cylinder 202 may be manufactured seamlessly to create an integrated part. Lock spring 524 may be disposed around lock pin 522. Lock spring 524 may be disposed base 514 and a pin plate 5222. Pin plate 5222 may be attached to lock pin 522. Pin plate 5222 and lock pin 522 may be manufactured seamlessly to create an integrated part. Lock spring 524 may be configured to apply an upward force to pin plate 5222. Lock spring 524 may be configured to urge lock pin 522 to move upward inside lock hole 512 by applying an upward force to pin plate 5222.

As further shown in FIG. 5C, plurality of pin receiving holes 526 may be provided at a bottom end 542 of adjustment cylinder 402. Each of plurality of pin receiving holes 526 may be configured to receive lock pin 522. Lock pin 522 may be configured to prevent or otherwise minimize rotational movement of adjustment cylinder 402 around rotation axis 422 when lock pin 522 is present in a pin receiving hole from plurality of pin receiving holes 526.

Lock pin 522 may include a handle 5224 attached to pin plate 5222. A user may disengage lock pin 522 from adjustment cylinder 402 by pulling down handle 5224 in a first direction 525. When lock pin 522 is pulled down inside lock hole 512 and lock pin 522 is disengaged from adjustment cylinder 402, a user may be able to rotate adjustment cylinder 402 around rotation axis 422. When handle 5224 is released, lock spring 524 may push up lock pin 522 in a second direction 527. When lock pin 522 is pushed up in second direction 527, lock pin 522 may be inserted in a pin receiving hole from plurality of pin receiving holes 526. When lock pin 522 is inserted in a pin receiving hole from plurality of pin receiving holes 526, lock pin 522 may engage with adjustment cylinder 402 and, thereby, rotational movement of adjustment cylinder 402 around rotation axis 422 may be prevented.

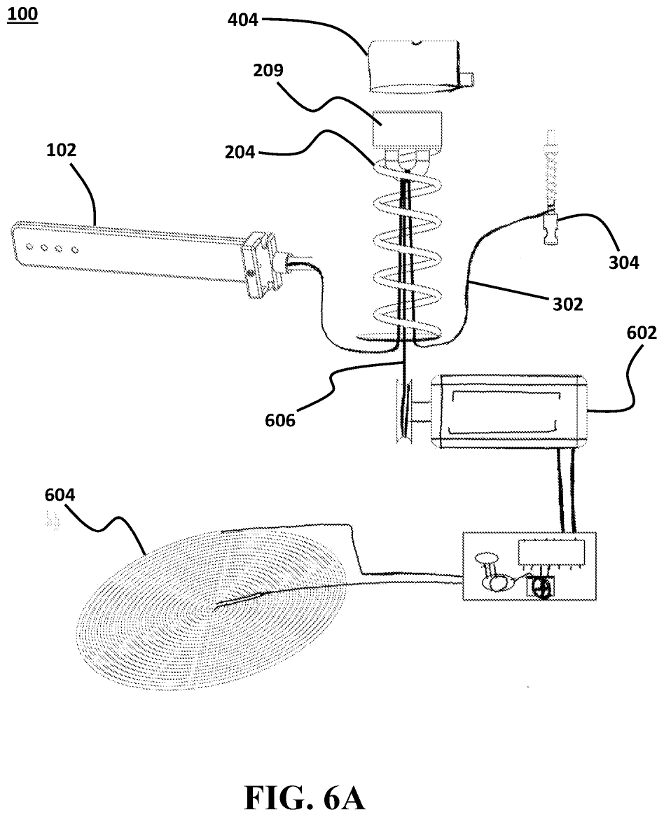

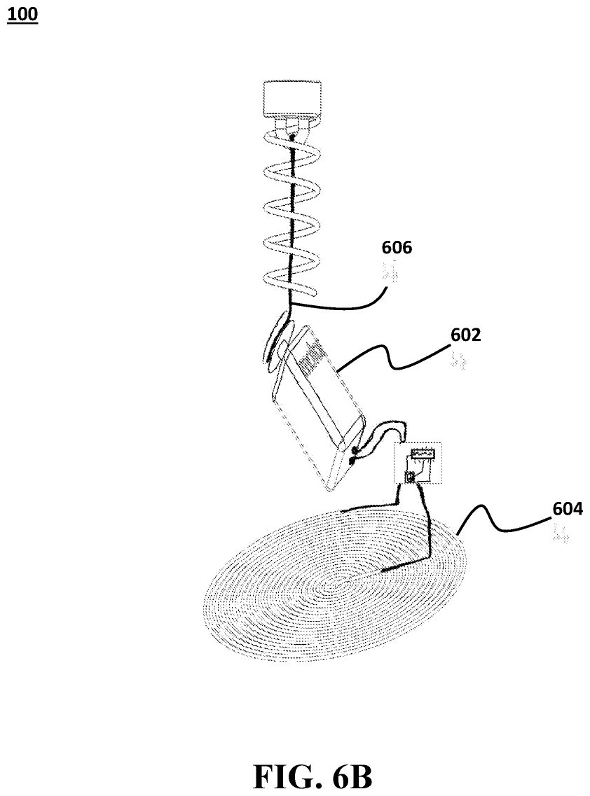

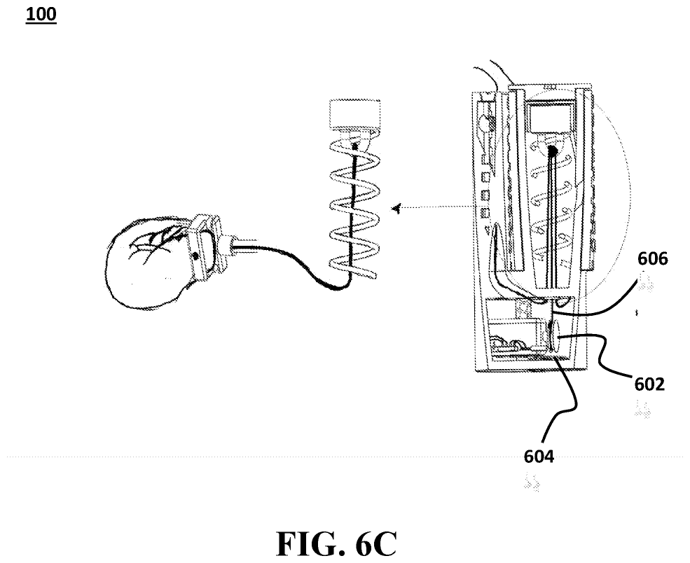

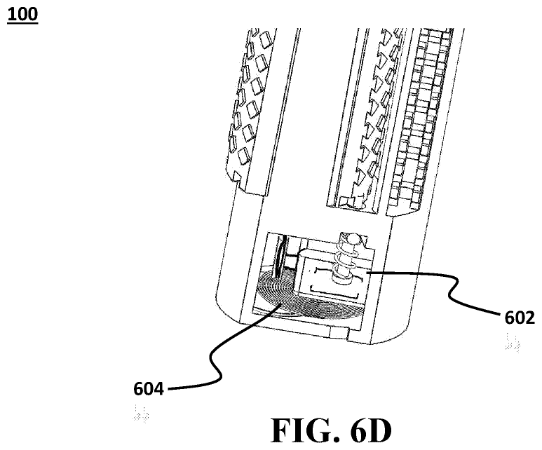

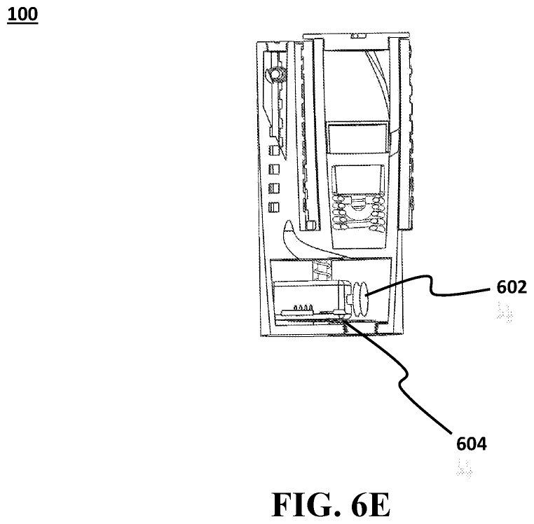

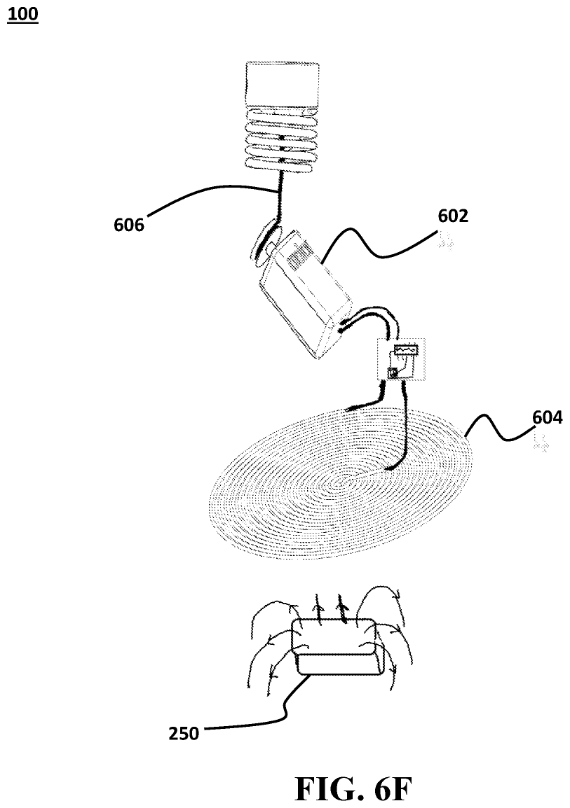

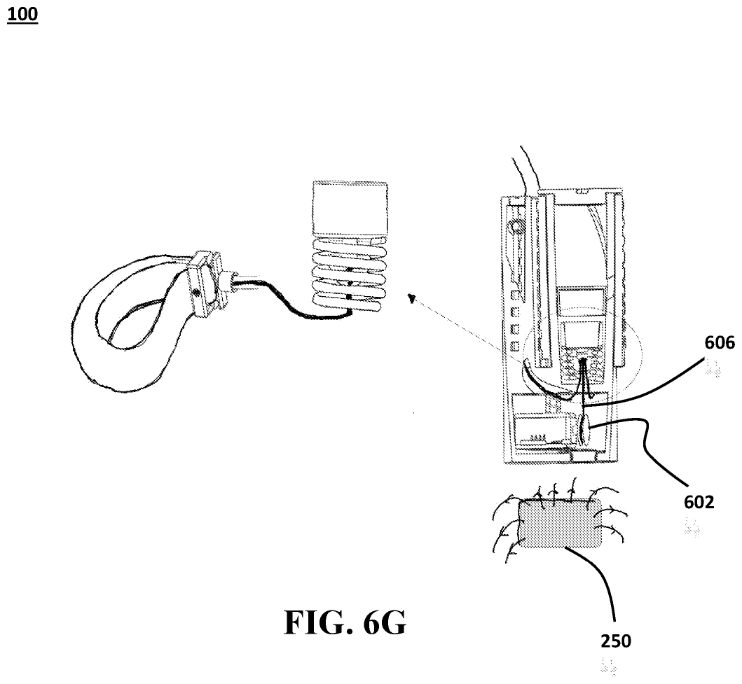

FIGS. 6A-6E show artificial urethral sphincter 100, consistent with one or more exemplary embodiments of the present disclosure. As shown in FIGS. 6A-6E, artificial urethral sphincter 100 may further include an electromotor 602 and a solenoid 604. Electromotor 602 may be disposed under hollow cylinder 202. A pull cable 606 may be interconnected between electromotor 602 and moveable part 209. Electromotor 602 may be configured to pull second end 244 of spring 204 toward first end 242 of spring 204 when electromotor 602 rotates in a first rotational direction. Electromotor 602 may further be configured to release second end 244 of spring 204 when electromotor 602 rotates in a second rotational direction. Solenoid 604 may be in connection with electromotor 602. Solenoid 604 may be configured to provide an induction current for electromotor 602. When the magnet 250 moves toward and close to solenoid 604, electromotor 602 may rotate in the first rotational direction and, to thereby, may pull second end 244 of spring 204 toward first end 242 of spring 204. Magnet 250 may include a magnetic part, a magnetic inductor, an electromagnetic inductor, or a combination thereof. When spring 204 has a large spring stiffness constant, electromotor 602 may be used to pull second end 244 of spring 204 toward first end 242 of spring 204.

FIG. 6F shows artificial urethral sphincter 100 in a scenario in which second end 244 of spring 204 is pulled toward first end 242 of spring 204, consistent with one or more exemplary embodiments of the present disclosure. FIG. 6G shows artificial urethral sphincter 100 in a scenario in which second end 244 of spring 204 is pulled toward first end 242 of spring 204, consistent with one or more exemplary embodiments of the present disclosure.

In an exemplary embodiment, artificial urethral sphincter 100 may further include a controller and a microchip in connection with the electromotor. The controller and the microchip may be configured to control movements of electromotor 602. The controller and the microchip may be in connection with a Bluetooth or wireless module, which may enable the controller and the microchip to be programmed when artificial urethral sphincter 100 is implanted inside patient's 110 body. A size of cuff member 102 and the amount of stretch in cable 206 may be adjusted independently through programming. The controller and the microchip may help artificial urethral sphincter 100 to be developed with artificial intelligence.

In an exemplary embodiment, artificial urethral sphincter 100 may be used as an artificial sphincter for anal sphincter and/or lower esophagus sphincter.

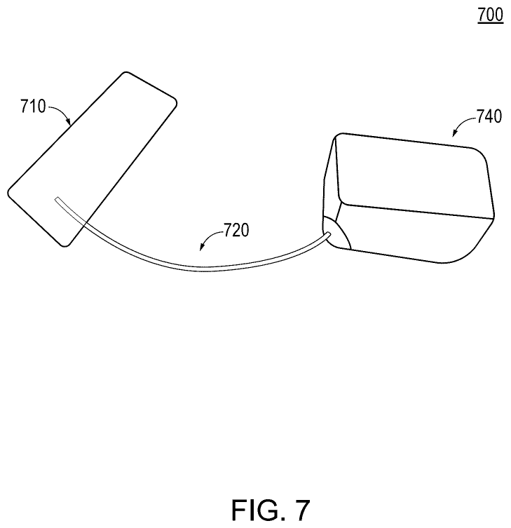

FIG. 7 shows an artificial sphincter 700 (hereinafter, also sometimes abbreviated to sphincter) according to some embodiments. Sphincter 700 includes a cuff 710, an actuator device (actuator 740), and a connection part 720 that connects actuator 740 to cuff 710.

In some embodiments, sphincter 700 may be an implantable device that can be placed inside the body of a patient. When sphincter 700 is implanted, cuff 710 may wrap around the urethra of the patient. Moreover, connection part 720 may include various parts such as a cable assembly, as further detailed below. Actuator 740 may include mechanisms for contracting (or closing) cuff 710 by tightening (“pulling,” that is, increasing the tension in) one or more cables that are inside connection part 720. Actuator 740 may further include mechanisms for expanding (or opening) cuff 710 by loosening (“releasing”, that is, reducing the tension in) one or more of those cables. Details of the design and function of these parts are further described below.

In some embodiments, cuff 710 may wrap around different types of lumens in the body (such as urethra, arteries, veins, colons, etc.). Therefore, sphincter 700 may be utilized to control in such lumens the flow of the corresponding bodily liquid (such as urine or blood) or more generally the movement of the corresponding bodily substance (such as feces moving inside the colon, hereinafter in this disclosure such substances also included in the generalized term bodily liquid). Similarly, cuff 710 may wrap around body parts that include one or more lumens such that contracting or expanding the cuff controls the blockage of those lumens. For example, cuff 710 may wrap around one or more of the limbs to block bleeding through one or more of the veins in case of injuries, therefore functioning like a controllable tourniquet.

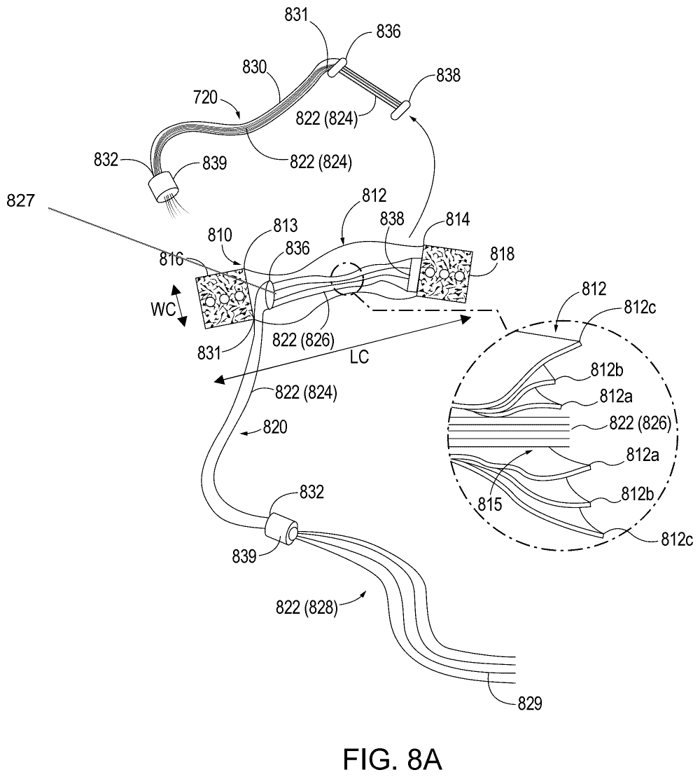

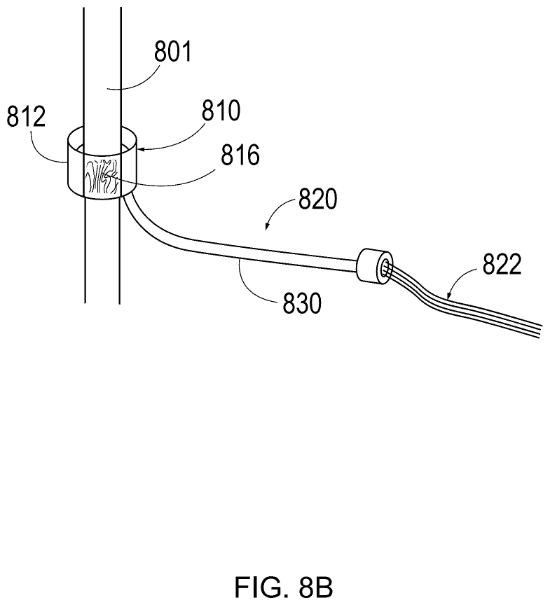

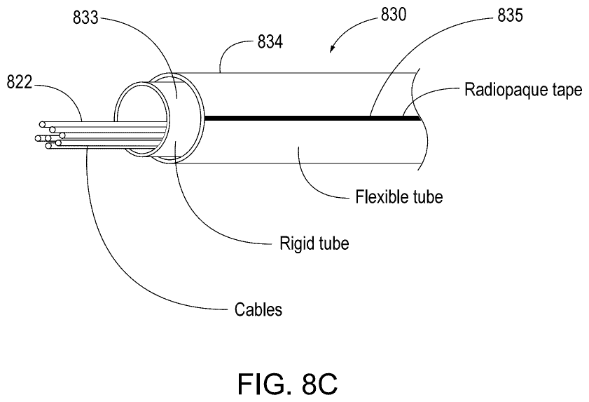

FIGS. 8A-8C show some details of a cuff 810 and a connection part 820 according to some embodiments. As generally stated above for similarly named parts, cuff 810 and connection part 820 may respectively be similar to cuff 710 and connection part 720 of FIG. 7. Cuff 810 includes a cuff body 812, an internal pocket 815 disposed in cuff body 812, a first attaching member 816, and a second attaching member 818.

Cuff body 812 itself has a proximal end 813 and a distal end 814. In some embodiments, cuff body 812 may be constructed of multiple layers. For example, in the embodiment illustrated in FIG. 8A, cuff body 812 includes an inner layer 812a, a middle layer 812b disposed outside of inner layer 812a, and an outer layer 812c disposed outside of middle layer 812b.

Inner layer 812a may be made of a material that is more rigid than the material of middle layer 812b and the material of outer layer 812c. This rigidity may be utilized to prevent excessive deformation of cuff body 812 and to prevent damaging cables 822, which are partially disposed in internal pocket 815 of cuff 810. Middle layer 812b may be made of a soft, flexible material, such as silk, to facilitate controlled deformation of cuff body 812. Outer layer 812c, which may come into contact with the internal organs or the skin, may be made of a flexible, biocompatible material. For example, in some embodiments, inner layer 812a may be made of nylon, middle layer 812b may be made of silk, and outer layer 812c may be made of silicone. However, other materials may be used for one or more of these layers, material such as polyurethane, alginate, polyamide, etc.

In some embodiments, cuff 810 may have a total length (LC) around 2-8 cm and a total width around 1-3 cm. However, cuff 810 is not limited to any particular dimensions, and the dimensions may vary depending on a particular application.

Cuff 810 may be configured to encircle a urethra of a patient, for example. As shown in FIG. 8B, when cuff 810 encircles a urethra 801, cuff 810 may form a closed loop around urethra 801, such that proximal end 813 and distal end 814 of cuff body 812 are secured together or are adjacent to each other. First and second attaching members 816 and 818 may be attached to each other when the cuff is configured in the closed loop and placed around the urethra. For example, in some embodiments, first and second attaching members 816 and 818 may be sutured, glued, or otherwise fastened to each other to secure cuff 810 in the form of a closed loop. In some embodiments, first and second attaching members 816 and 818 may be made of a mesh material that facilitates attachment of first and second attaching members 816 and 818 to each other.

Connection part 820 includes cables 822, a cable jacket 830 having a first end 831 adjacent to cuff 810 and a second end 832 adjacent to actuator device 740, a proximal cable support 836, a distal cable support 838, and a connector 839 attached to second end 832 of cable jacket 830.

In some embodiments, four or more cables 822 may be provided. However, fewer or more than four cables 822 may be provided in other embodiments. In some embodiments, cables 822 may each be a filament or a spun fiber. For example, cables 822 may be nylon monofilaments, while other materials and constructions are also possible. Middle segments 824 of cables 822 extend inside cable jacket 830 between cuff 810 and actuator 740. First end segments 826 of cables 822 are disposed in internal pocket 815 of cuff 810. First end segments 826 extend through openings in proximal cable support 836 and include first ends 827 of cables 822 that are fixed to distal cable support 838.

Proximal cable support 836 is disposed inside cuff body 812, adjacent to first end 831 of cable jacket 830, and is attached to proximal end 813 of cuff body 812. Distal cable support 838 is disposed inside cuff body 812 and is attached to distal end 814 of cuff body 812.

Connector 839 of connection part 820 may be connected to actuator 740. For example, connector 839 may be a threaded connector configured to connect to a threaded portion of actuator 740.

Second end segments 828 of cables 822 extend from second end 832 of cable jacket 830 and terminate at second ends 829 of cables 822. Second end segments 828 may be disposed inside actuator 740, where the second end segments 828 may engage cuff actuating components of actuator device 740 that are operable to control (e.g., pull and release) cables 822 to selectively contract or release cuff 810. Those cuff actuating components will be described in detail below.

FIG. 8C shows some more details of cable jacket 830 according to some embodiments. More specifically, cable jacket 830 may include an inner jacket 833 and an outer jacket 834. Inner jacket 833 may be a tube made of a material that is more rigid than a material of outer jacket 834, so as to prevent cables 822 from excessively bending and binding therein. For example, inner jacket 833 may be made of a thermoplastic material. Outer jacket 834 may be a tube made of a material that is softer and more flexible than the material of inner jacket 833, to improve flexibility of connection part 820 and prevent discomfort in an area of a person's body in which the sphincter is disposed. For example, outer jacket 834 may be made of polyurethane. Additionally, in some embodiments, a radiopaque tape 835 may be disposed on or in outer jacket 834 to facilitate identification of connection part 820 in the person's body using an x-ray machine or a fluoroscope.

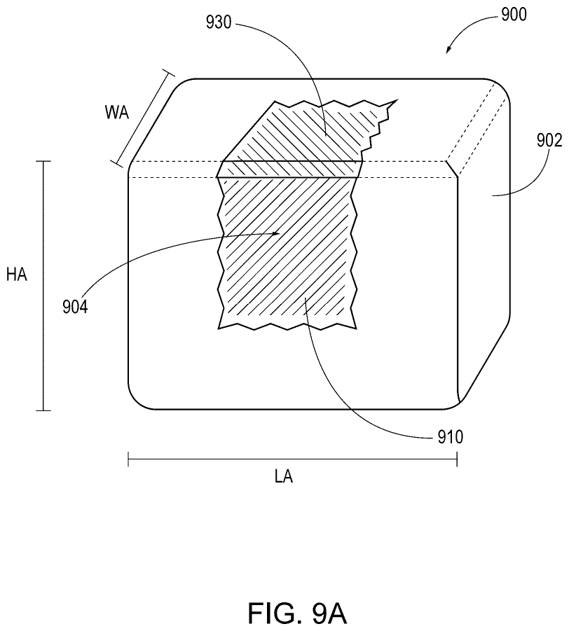

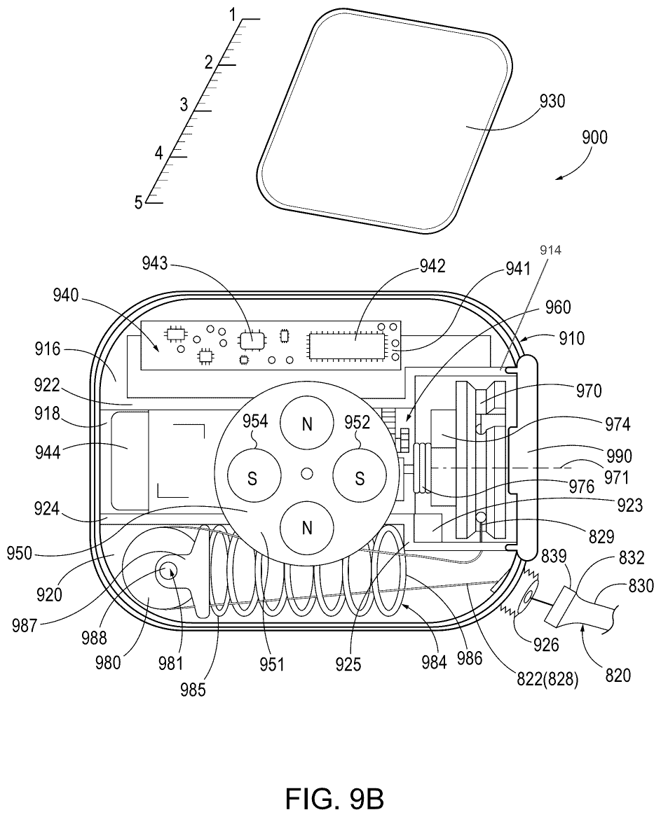

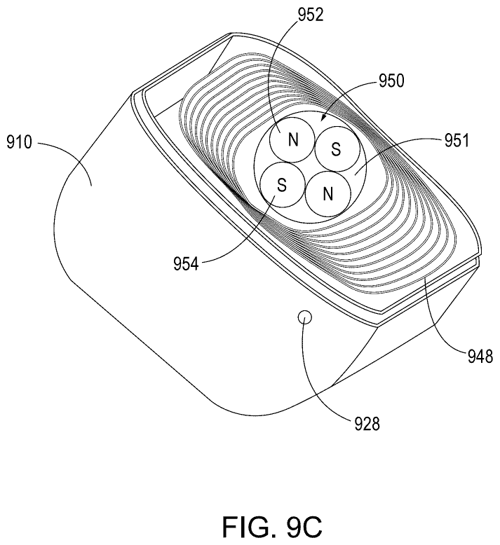

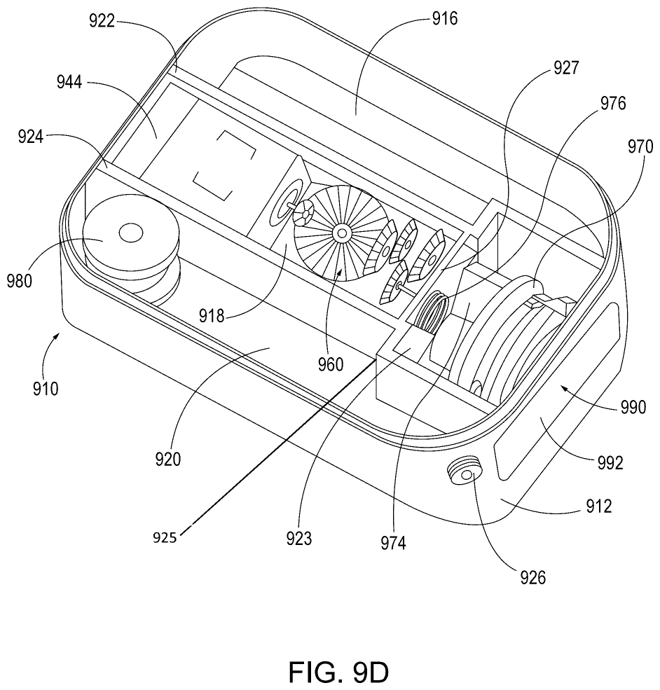

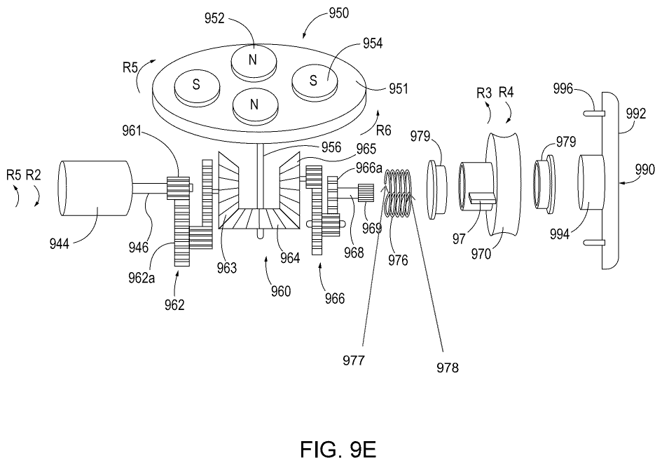

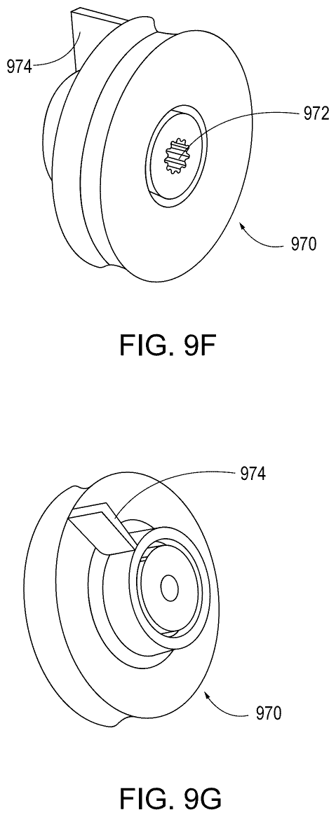

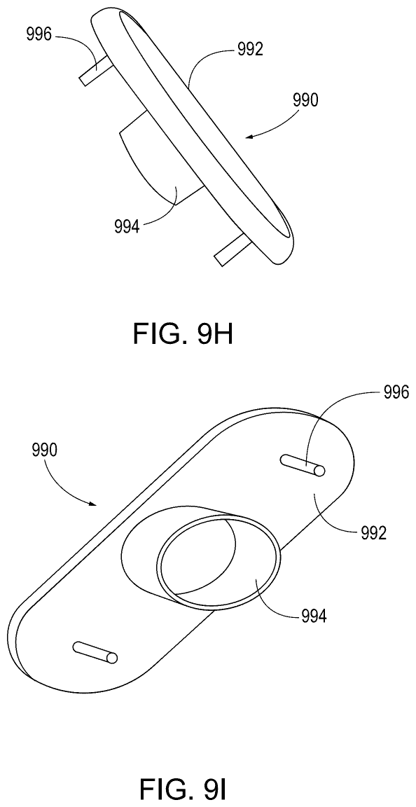

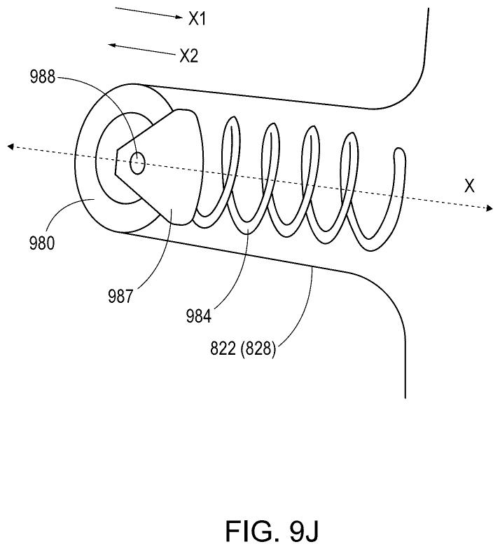

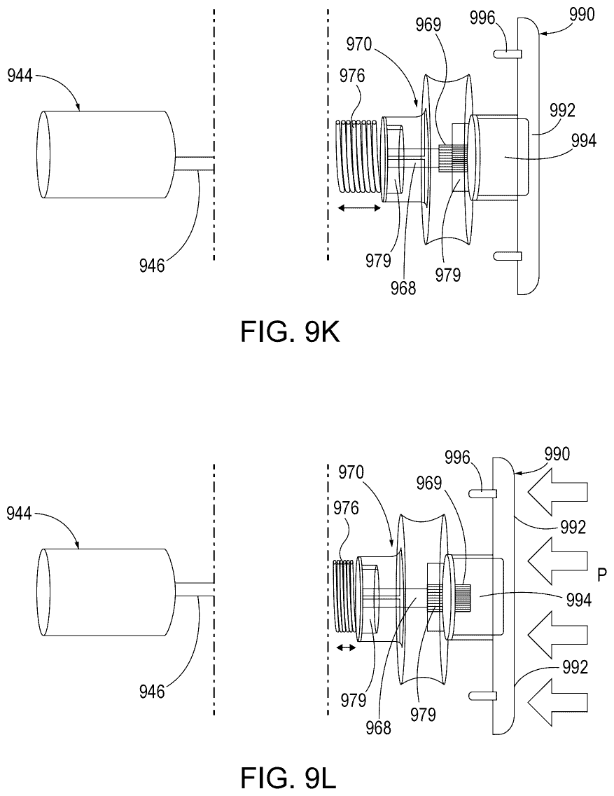

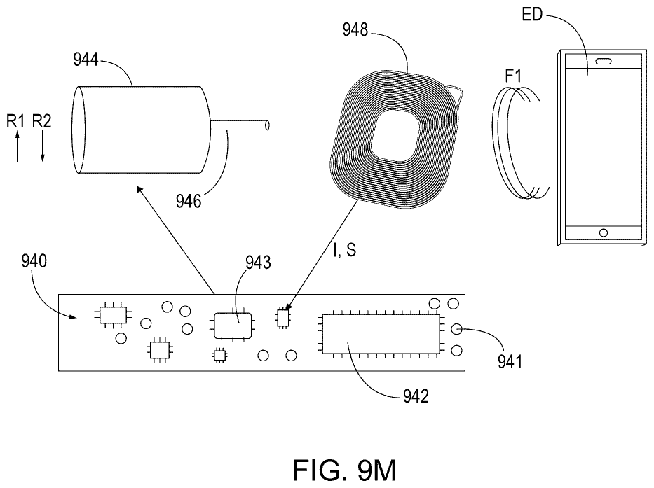

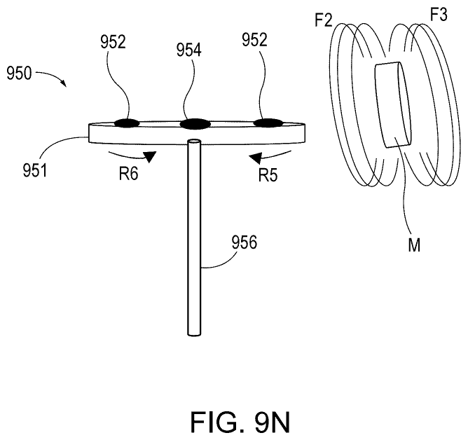

FIGS. 9A-9G show the detailed structure of an actuator 900 according to some embodiments. As stated above more generally for similarly named parts, actuator 900 may be similar to actuator device 740 of FIG. 7.

FIG. 9A shows an outer structure of actuator 900 according to some embodiments. Actuator 900 includes a housing 904 containing cuff actuating components, and an outer shell 902 covering housing 904. In various embodiments, outer shell 902 may seal the interior of housing 904 from external liquids and other elements. In some embodiments, outer shell 902 may be made of a biocompatible material such as silicone. In some embodiments, housing 904 may be made of a biocompatible synthetic material such as polyurethane or polyamide.

Housing 904 includes a base 910 and a cover 930 attached to base 910 to enclose the interior of housing 904. In some embodiments, outer shell 902 and cover 930 can be opened or removed from base 910 to provide access to the cuff actuating components.

In some embodiments, actuator 900 may have a length LA of approximately 4 cm to approximately 8 cm, a width WA of approximately 2 cm to approximately 8 cm, and a height HA of approximately 4 cm to approximately 8 cm. However, actuator 900 is not limited to any particular dimensions, and the size of actuator 900 may vary according to an application.

FIGS. 9B-9G show some details of actuator 900. In FIGS. 9B-9D, outer shell 902 is omitted for illustrative purposes. As shown in FIGS. 9B-9E, the cuff actuating components may include an electronic board 940 (shown in FIG. 9B but omitted in FIG. 9D), a motor 944, a wire coil 948 (also called herein an induction coil or a magnetic induction coil; shown in FIG. 9C but omitted in FIGS. 9B and 9D to more completely show other components), a magnet assembly 950 (shown in FIGS. 9C and 9E, but omitted in FIG. 9D to more completely show other components), a gear assembly (or gearbox) 960 (shown in FIGS. 9B, 9D, and 9E), a first pulley 970, a first spring 976, a second pulley 980, a second spring 984 (omitted in FIG. 9D), and an emergency button 990.

In various embodiments, such as those shown in FIGS. 9A-9G, the actuator may include multiple mechanisms for operating the cuff. The multiple mechanisms may include, for example, one or more main mechanisms (used during normal operations of the sphincter) and one or more alternative (sometimes called backup or fail-safe) mechanisms, used when the main mechanism is not available or fails as further detailed below. For actuator 900, for example, the main mechanism may include motor 944 in combination of other parts. In some embodiments, motor 944 is a DC motor.

The alternative mechanisms (also called herein an alternative actuation mechanism), on the other hand, may include magnet assembly 950 in combination with other parts or emergency button 990 in combination with other parts, as further detailed below. In the main mechanism, a rotation of motor 944 is transmitted through gear assembly 960 to first pulley 970, which in turn causes tightening or loosening of the wires and operating the cuff. In the alternative mechanism, on the other hand, a rotation of magnet support 951 may replace the rotation of motor 944. The alternative mechanism utilizing emergency button 990, on the other hand, causes a rotation of first pulley 970 in a third manner detailed below.

In various embodiments, when the artificial sphincter uses a main mechanism of the actuator, the artificial sphincter, or its parts, are considered to be in an operational state. For example, in some embodiments, in the operational state the actuator is operational and performs its normal operations as described here. On the other hand, when the artificial sphincter uses an alternative mechanism (for various reasons that may include unavailability of the main mechanism, convenience of the alternative mechanism, or testing the alternative mechanism) the artificial sphincter, or its parts, are considered to be in a non-operational state. For example, in some embodiments, in a non-operational state the actuator may not be operational. These states are further described below.

One or more of the cuff actuating components are mounted in base 910. In some embodiments, electronic board 940, motor 944, wire coil 948, magnet assembly 950, gear assembly 960, first and second pulleys 970 and 980, first and second springs 976 and 984, and emergency button 990 may all be disposed in base 910. In some embodiments the wire coil may include a solenoid. Further, the wire coil may generate the induced current in response to a magnetic induction originated by a device located outside the body, herein also called a transcutaneous magnetic induction. In some embodiments, the magnetic induction may cause a temporally varying magnetic flux in the induction coil.

In some embodiments, as illustrated in FIGS. 9B and 9D, for example, base 910 includes a first compartment 916, a second compartment 918, and a third compartment 920. First and second compartments 916 and 918 are separated from each other by a first interior divider wall 922, and second and third compartments 918 and 920 are separated from each other by a second interior divider wall 924.

Electronic board 940 is disposed in first compartment 916. Motor 944, gear assembly 960, first pulley 970, and first spring 976 are disposed in second compartment 918. Emergency button 990 is disposed in a sidewall 912 of base 910 and extends into second compartment 918. Second pulley 980 and second spring 984 are disposed in third compartment 920. Wire coil 948 and magnet assembly 950 are disposed in a space above first, second, and third compartments 916, 918, and 920.

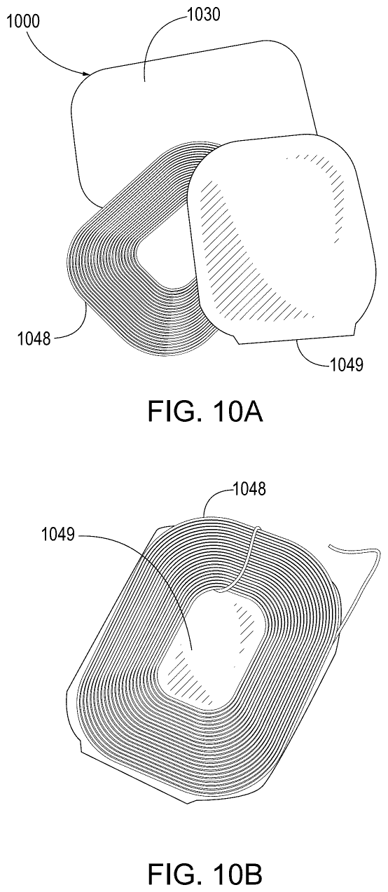

However, the arrangement of components illustrated in FIG. 9B is merely an example and, in some embodiments, one or more of the cuff actuating components may be mounted in cover 930. For example, FIGS. 10A and 10B illustrate a cover assembly 1000 according to some other embodiments. Cover assembly 1000 includes a cover 1030, a wire coil 1048, and a magnetic shield 1049. These components may therefore replace cover 930 and wire coil 948 of FIGS. 9A to 9C. In cover assembly 1000, wire coil 1048 may be mounted on an inner surface of cover 1030 and magnetic shield 1049 may be disposed over wire coil 1048 opposite cover 1030. Magnetic shield 1049 may shield wire coil 1048 from electrical interference by other components of an actuator, and may further shield other components of the actuator from external electrical interference as well as electromagnetic interference from wire coil 1048. A magnetic shield similar to magnetic shield 1049 may also be included on cover 930 of FIGA. 9A and 9B.

Further, in other embodiments, a wire coil similar to wire coils 948 and 1048 of FIGS. 9C and 10A-10B, respectively, may be disposed in the patient's body. These wire coils may be placed separately or away from the other components of actuator 900 (e.g., outside of outer shell 902/housing 904) such that the wire coil may be located closer to the patient's skin.

Referring back to FIG. 9B, electronic board 940 may be electrically connected to motor 944 and wire coil 948, via, for example, one or more electric wires. Moreover, electronic board 940 may be configured to receive power through a varying magnetic or electromagnetic field applied to wire coil 948. In various embodiments, the varying electromagnetic field may be an electromagnetic field for which the magnitude consistently increases, consistently decreases, or alternates during a time period. Further, electronic board 940 may be configured to drive motor 944, using electricity induced by, or based on data transmitted through, the electromagnetic field applied to wire coil 948, to selectively contract or expand cuff 810 around urethra 801. For example, electronic board 940 may be configured to selectively rotate motor in a first direction (e.g., clockwise) to contract cuff 810 or rotate motor 944 in a second direction (e.g., counterclockwise) to expand cuff 810, or vice-versa. The selection of one or more of the operations, such as direction of rotation or amount of rotation, may depend on one or more characteristics of the electromagnetic field or, accordingly, one or more characteristics of the induced electricity transmitted from wire coil 948 to electronic board 940.

In some embodiments, electronic board 940 may include a rectifier 941, a controller 942, and a motor driver 943. In some embodiments, rectifier 941 may be configured to output a rectified (DC) current from an induced current that is induced by the electromagnetic field through wire coil 948. Electronic board 940 may be powered by the rectified current. The rectified current may also drive motor 944. Moreover, controller 942 may be configured to control motor driver 943 to drive motor 944 according to the data transmitted through the electromagnetic field. Controller 942 may derive the data from one or more characteristics of the rectified current. Those characteristics may include, for example, the duration or the magnitude of the rectified current. The data may also be derived based on a combination of the characteristics of a sequence of rectified currents received by controller 942, as further detailed below.

In some embodiments, electronic board 940 and motor 944 may be entirely powered by the electromagnetic field applied to wire coil 948. That is, in some embodiments, the rectified current generated by rectifier 941 may supply power to controller 942 and motor driver 943, and motor driver 943 may apply a driving voltage to motor 944 from the rectified current. Accordingly, actuator 900 may not require a battery as a power source, thus eliminating one or more of the burdens or problems posed by requiring such a battery. For example, use of a battery may necessitate mechanisms or procedures for occasional replacement of the battery when the battery is discharged or malfunctions. Battery replacement, in turn, may require an invasive procedure to remove the actuator from the body of the patient.

In some embodiments, controller 942 may store or receive data from a user and accordingly operate the actuator. The user may transmit the data by controlling the changes in the electromagnetic field in the wire coil or controlling changes in a source of alternating electromagnetic field. In order to store or receive data, controller 942 may include one or more storages for storing data including operating parameters or information such as a cuff state information, one or more memories for storing instructions for interpreting received information and controlling operation of motor driver 943, and one or more processors configured to execute the stored instructions.

The cuff state information may include, for example, information regarding whether cuff 810 is currently tightened so as to close urethra 801 or is currently loosened so as to allow urethra 801 to be open. The cuff state information may be obtained by one or more position sensors or position calculators connected to electronic board 940 and configured to detect a position of one or more of a motor shaft 946 (FIG. 9E), an output shaft 968 (FIG. 9E) of gear assembly 960, and first pulley 970. Additionally or alternatively, the cuff state information may include information on a direction in which motor 944 rotated in one or more of the most recent operations of motor 944, or may use a state variable that switches between a closed estate and an open state with each change of state, and in this manner keep track of the cuff state at any time.

In some embodiments, the cuff open/close-state is related to rotational performance of the motor, and is controlled within a 360-degree range using a mechanical pin. The distance between these 360 degrees may be monitored by position check sensors, such as optocounter sensors. Accordingly, the tension may be adjusted based on the motor torque calibration based on the received voltage and applied tension force to the cable. In some embodiments, based on laboratory experiments with different voltage applications to the motor, different torques are generated within specific ranges. The system may rotate within a 360-degree range by creating constant pressure.

The one or more processors of controller 942 may execute the stored instructions to interpret the data received by controller 942 through the electromagnetic field applied to wire coil 948, and to control motor driver 943 based on that data. In various embodiments, some operational information may be stored on a chipset included in the controller. The operational information may include activation frequency, motor torque, speed, and notifications sent, and the cuff state information.

For example, controller 942 may receive information included in the induced current or the rectified current. The information may include the duration of the induced current or the rectified current. This situation may occur when, for example, an operator or the user holds an external inducer (defined here as a source of alternating magnetic field located outside the body) near the wire for the first range of the duration of time. The inducer may include a wearable device that uses a varying magnetic field to activate the actuator. Upon this activation, the actuator may signal the electromotor to perform tasks based on a set program. The tasks may include mechanical functions (e.g., opening or closing the cuff) or adjusting some settings of the sphincter.

In various embodiments, the artificial sphincter may operate with two different types of induction mechanisms. The first type may include using a permanent magnet and a second type may include using a source of varying magnetic field. In some embodiments, the permanent magnet may be used to move some parts inside the sphincter. The varying magnetic field, on the other hand, may induce an electric current in the coil, causing movement of some parts. Alternatively, or in addition, the induced current may be utilized to transfer data to the sphincter.

FIG. 9E shows the mechanical connection assembly of the drive systems, according to some embodiments. The mechanical connection assembly includes motor 944, magnet assembly 950, and gearbox emergency release button 990, which acts like a clutch. Motor 944 may be connected to gear assembly 960 by motor shaft 946, which is rotated by rotation of motor 944. The mechanical connection assembly may allow the units to operate independently and may directly connect to the main pulley 970. Moreover, in some embodiments, such as the one shown in FIG. 9E, while motor 944 and magnet assembly 950 are able to rotate the main pulley (respectively through first driving gear 961 and second driving gear 964), they are not able to rotate each other.

In some embodiments, wire coil 948 is a flat wire coil, such as coil 604 in FIG. 6F or wire coil 948 in FIG. 9C. However, other types of wire coils, such as a helical wire coil, may be provided in other embodiments. Wire coil 948 may be made of copper or another conductive material suitable for providing an induced current when exposed to an electromagnetic field.

As explained above, magnet assembly 950 and gear assembly 960, together, may provide an alternative mechanism to motor 944 for rotating first pulley 970. More specifically, in some embodiments, a rotation of magnet assembly 950 may be transferred to first pulley 970 through gear assembly 960. Some details of this alternative mechanism and each of its parts is now explained.

First, regarding the magnet assembly, FIGS. 9B, 9C, and 9E show that, in some embodiments, magnet assembly 950 includes a magnet support 951, a first set of (in this case two) magnets 952 having a first polarity, and a second set of (in this case two) second magnets 954 having a second polarity opposite to the first polarity. First and second magnets 952 and 954 may be arranged in alternating order on magnet support 951. However, in other embodiments, a single magnet may be used instead of first and second magnets 952 and 954. As shown in FIG. 9E, magnet assembly 950 is connected to gear assembly 960 by magnet assembly output shaft 956 that is connected to magnet support 951. As will be described later in more detail, magnet assembly 950 may be driven by a an externally applied magnetic field.

Next, regarding the gear assembly, FIG. 9E shows that, in some embodiments, gear assembly 960 includes a first driving gear 961, a first group of driven gears 962, a first bevel gear 963, a second driving gear 964, a second bevel gear 965, a second group of driven gears 966, the output shaft 968, and an output gear 969. As detailed here, the gear assembly may be involved, in part or in full, in one or more of the main mechanisms and the alternative mechanisms. For example, in actuator 900, gear assembly 960 is utilized both in the main mechanism and the alternative mechanism utilizing magnet support 951.

First driving gear 961 and output gear 969 may be pinion gears. The second driving gear 964 may be a bevel gear. The first and second groups of driven gears 962 and 966 may include spur gears. First driving gear 961 may be mounted on motor shaft 946 and may be configured to rotate with motor shaft 946. First driving gear 961 may mesh with a first driven gear 962a in the first group of driven gears 962. First bevel gear 963 may be configured to rotate with rotation of the first group of driven gears 962. Second driving gear 964 may be mounted on magnet assembly output shaft 956 and may mesh with first bevel gear 963 and second bevel gear 965. The second group of driven gears 966 may be configured to rotate with rotation of second bevel gear 965. A final gear 966a in the second group of driven gears 966 may be mounted on one end of output shaft 968, and output gear 969 may be mounted on another end of output shaft 968. Thus, output shaft 968 and output gear 969 may be configured to rotate together with final gear 966a in the second group of driven gears 966.

Next, regarding the first pulley, FIGS. 9B and 9D to 9G show that, in some embodiments, in an operational state, first pulley 970 is rotatably connected to gear assembly 960 and may have a rotational axis 971. More specifically, in some embodiments, in the operational state, output gear 969 is received in first pulley 970 and meshes with internal teeth 972 (FIG. 9F) of first pulley 970 such that first pulley 970 is configured to rotate in response to rotation of output shaft 968.

First spring 976 (shown, for example, in FIGS. 9B, 9D, 9K, and 9L) is a compressible spring that causes actuator 900 to be in the operational state. First spring 976 may be disposed between gear assembly 960 and first pulley 970, and first pulley 970 may be disposed between first spring 976 and emergency button 990. As shown in FIG. 9D, first end 977 of first spring 976 may be supported by a support wall 927 of base 910. Moreover, as shown in FIG. 9E, a second end 978 of first spring 976 may interface with the first side of first pulley 970 through a washer 979. Moreover, as also shown in FIG. 9E, a second side of first pulley 970 may be in contact with emergency button 990 through another washer 979.

Because of this configuration, in the operational state, first spring 976 may maintain 970 at a distance from support wall 927 such that output gear 969 remains engaged with internal teeth 972.

For actuator 900 to transition into the non-operational state, on the other hand, a pressure may be exerted on emergency button 990 causing first pulley 970 to move closer to support wall 927 while compressing first spring 976. This displacement of first pulley 970 toward support wall 927, if large enough, may cause internal teeth 972 to disengage from 969, thus allowing first pulley 970 to rotate freely if, for example, the wire wrapped around first pulley 970 is pulled by a tension. The details of this transition are further described below with respect to FIGS. 9K and 9L.

In some embodiments, first pulley 970 includes a stop arm 974 disposed on a first side thereof to limit rotation of first pulley 970. For example, stop arm 974 may be configured to engage a projection 923, located on second interior divider wall 924 that extends into second compartment 918 to stop rotation of first pulley 970. In some embodiments, stop arm 974 and projection 923 may limit rotation of first pulley 970 to approximately a full rotation (360°).

Next, second pulley 980 (shown, for example, in FIGS. 9B and 9J) is utilized for supporting cables 822 in its path between first pulley 970 and the cuff. Moreover, in some embodiments, the combination of second pulley 980 and second spring 984 may provide a suspension mechanism for maintaining cables 822 taut and still providing a safety mechanism for some special situations, further described later. As shown in FIG. 9B, second pulley 980 may be rotatably mounted on a shaft 988 of a pulley mount 987 that is attached to or integrally formed with a first end 985 of second spring 984. Moreover, second pulley 980 may have a rotational axis 981 that is orthogonal to rotational axis 971 of first pulley 970. First end 985 of second spring 984, pulley mount 987, and shaft 988 are not fixed to base 910. In other words, first end 985 of second spring 984, pulley mount 987, and shaft 988 are movable within third compartment 920. As shown, for example, in FIGS. 9B and 9D, second end 986 of second spring 984 may be seated on a ledge 925 of second interior divider wall 924. Second end segments 828 of cables 822 may be disposed inside base 910. More specifically, second end segments 828 of cables 822 may extend from second end 832 of cable jacket 830 into base 910 through a cable port 926 in sidewall 912 of base 910, around second pulley 980 in third compartment 920, and onto first pulley 970 in second compartment 918. In some embodiments, second ends 829 of cables 822 may be fixed to first pulley 970. Cable port 926 is exposed through outer shell 902 and may be connected to connector 839, which is attached to cable jacket 830, to seal cable port 926 from external fluids. For example, cable port 926 may include threads configured to engage threads of connector 839.

FIGS. 9H and 9I illustrate some details of emergency button 990. As shown in FIGS. 9H and 9I, in some embodiments, emergency button 990 includes a plate portion 992, a projecting portion 994 projecting from an inner surface of plate portion 992, and guideposts 996 projecting from the inner surface of plate portion 992. As will be described later in more detail with respect to FIGS. 9H and 9I, plate portion 992 is configured to be pressed inward by the user's finger, the user's thumb, or an object held by the user, and projecting portion 994 is configured to press against first pulley 970 to disengage first pulley 970 from output shaft 968/output gear 969 when plate portion 992 is pressed. Guideposts 996 are received in openings 914 in base 910 for guiding movement of emergency button 990 as the user presses it.

Actuator 900 may be operated to contract (sometimes also termed to close or to tighten) cuff 810 by tightening (“pulling,” that is, increasing the tension in) cables 822. Additionally, actuator 900 may be operated to expand (sometimes also termed to open, to loosen or to release) cuff 810 by loosening (“release,” that is, reducing the tension in) cables 822. When cuff 810 is formed in a loop. Around urethra 801 (as illustrated in FIG. 8B) and is contracted by actuator 900, an inner diameter of cuff 810 is decreased such that cuff 810 increases pressure applied to urethra 801 by cuff 810, thereby increasing restriction of urine flow or preventing urine flow through urethra 801. In contrast, when cuff 810 is formed in a loop around urethra 801 and is expanded by actuator 900, the inner diameter of cuff 810 is increased such that cuff 810 reduces pressure applied by to urethra 801 by cuff 810, thereby enabling urine flow or reducing restriction of urine flow through urethra 801. In some embodiments, releasing the cables causes expansion of the cuff due to an outward pressure of the liquid present in the urethra or the elasticity of urethra. In other embodiments, other mechanisms may also contribute to the expansion of the cuff, mechanisms such as an elasticity of cuff or its components.

Tightening or loosening of cables 822 to respectively contract or release cables 822 as described above may be performed by rotation of motor 944, by rotation of magnet assembly 950, or by other main or alternative mechanisms described here. Example operations of components of actuator 900 to contract and expand cuff 810 are described below with respect to FIGS. 9B to 9E.

For example, referring to FIG. 9E, motor 944 and motor shaft 946 may rotate in either a clockwise direction R1 or a counterclockwise direction R2 (as viewed from right to left in FIG. 9E) in response to an electromagnetic field that is selectively applied to wire coil 948 (FIG. 9C). When viewed from right to left in FIG. 9E, first pulley 970 may rotate in a clockwise winding direction R3 or a counterclockwise unwinding direction R4 (or vice-versa, depending on a configuration of gear assembly 960) in response to the clockwise rotation or counterclockwise rotation, respectively, of motor 944 and motor shaft 946. Referring to FIGS. 9B and 9E, when first pulley 970 rotates in the clockwise winding direction R3, cables 822 are wound around first pulley 970 and pulled over second pulley 980, thereby tightening cuff 810. Conversely, when first pulley 970 rotates in the counterclockwise unwinding direction R4, cables 822 are unwound from first pulley 970 and extended over second pulley 980, thereby loosening cuff 810. Operation of motor 944 to tighten or loosen cuff 810 may be controlled by controller 942. Example embodiments in which controller 942 controls operation of motor 944 are described later with respect to FIG. 9M.

Referring again to FIGS. 9B to 9E, magnet assembly 950 may also be operated to contract and expand cuff 810 around urethra 801. For example, referring to FIG. 9E, magnet assembly 950 and magnet assembly output shaft 956 may rotate in either a clockwise direction R5 or a counterclockwise direction R6 (as viewed from above in FIG. 9E) in response to a magnetic field that is selectively applied to magnet assembly 950. When viewed from right to left in FIG. 9E, first pulley 970 may rotate in the clockwise winding direction R3 or the counterclockwise unwinding direction R4 (or vice-versa, depending on a configuration of gear assembly 960) in response to the clockwise rotation or counterclockwise rotation, respectively, of magnet assembly 950 and magnet assembly output shaft 956. As explained earlier, these rotations in turn may cause tightening or loosening of the cuff.

Next, one or more safety mechanisms are described according to some embodiments. For example, referring to FIG. 9B, actuator 900 may include mechanisms for preventing damage to urethra 801 and physical pain to the user, or preventing damaging or failure of components of actuator 900 and cables 822 when actuator 900 is operated to contract cuff 810. For example, in some embodiments, as discussed above, stop arm 974 of first pulley 970 and projection 923 limit rotation of first pulley 970, thereby limiting the degree to which cables 822 are pulled and contraction of cuff 810. Thus, stop arm 974 and projection 923 may prevent excessive squeezing of urethra 801 that could result in damage to urethra 801 and/or in the user experiencing physical pain. Further, there may be circumstances in which cuff 810 is unable to contract to compress urethra 801, such as when there is a blockage in urethra 801. In such circumstances, excessive pulling of cables 822 by actuator 900 may break cables 822, thereby rendering cables 822 and cuff 810 inoperable. By limiting the degree to which cables 822 are pulled, stop arm 974 and projection 923 may prevent over-tensioning and breakage of cables 822.

Another safety mechanism may utilize second pulley 980 and second spring 984, which may be configured to prevent over-tensioning of cables 822. For example, second pulley 980 may reduce tension in cables 822 in circumstances in which cuff 810 is unable to contract for compressing urethra 801, such as when there is a blockage in urethra 801. More specifically, in some embodiments, first end 985 of second spring 984, pulley mount 987, and shaft 988 are not fixed to base 910 as described above with respect to FIG. 9B. Accordingly, second pulley 980 is not fixed to base 910 and is movable within third compartment 920. For example, as shown in FIG. 9J, second pulley 980 may be configured to move (translate) in directions parallel and transverse to a longitudinal axis X of second spring 984. When an amount of tension exceeding a threshold tension is applied to cables 822 during pulling of cables 822 by actuator 900, cables 822 may press second pulley 980 against second spring 984 and compress second spring 984 in a first direction X1 parallel to longitudinal axis X, thereby moving second pulley 980 in first direction X1. Thus, because second pulley 980 is resiliently mounted on second spring 984, second spring 984 may reduce tension cables 822 when the tension in cables 822 exceeds the threshold tension, and thereby prevent breakage of cables 822. Further, when second spring 984 is compressed and tension in cables 822 is thereafter reduced, second spring 984 may urge second pulley 980 in a second direction X2 opposite direction X1 to maintain proper engagement between second pulley 980 and cables 822, thereby preventing binding and tangling of cables 822. In some embodiments, second spring 984 is compressed even when second pulley 980 is located in its original position in third compartment 920 (that is, its farthest location in the X2 direction). This normal state compression may thus cause cables 822 to always remain taut and prevent them from binding or tangling. The degree of normal state compression may not exceed a maximum threshold to avoid exerting excessive tension on the cables.

Some safety mechanisms may be used in circumstances in which actuator 900 malfunctions or is damaged, and therefore cannot properly control contraction and expansion of cuff 810 when cuff 810 is disposed around urethra 801. For example, actuator 900 may malfunction due to circumstances such as failure or malfunction of electronic board 940 or components thereof, failure or malfunction of motor 944, failure or binding of gear assembly 960, tangling or binding of cables 822 inside actuator 900, damage to any of the components or structure of actuator 900, or the presence of magnetic or electromagnetic interference that prevents proper operation of electronic board 940, motor 944, or magnet assembly 950. Referring back to FIG. 8B, if actuator 900 malfunctions or is damaged such that it cannot expand cuff 810 from the state of being contracted around urethra 801 to allow the user to urinate, or if motor 944 otherwise maintains cuff 810 in an excessively contracted state around urethra 801, the user may suffer serious injury and/or medical complications.

In some embodiments, as one of the safety mechanisms for the above-described circumstances, actuator 900 includes emergency button 990 to enable cuff 810 to be expanded if actuator 900 malfunctions or becomes inoperable due to damage while cuff 810 is contracted around urethra 801. Functionality of emergency button 990 according to some embodiments is described below with respect to FIGS. 9K and 9L.

As shown in FIG. 9K (and described above with respect to FIGS. 9B, 9C, and 9E-9G), in the operational state output gear 969 is received in first pulley 970 and meshes with internal teeth 972 of first pulley 970. Thus, during normal operation of actuator 900, first pulley 970 is configured to rotate in response to rotation of output shaft 968.

Moreover, in the operational state, first pulley 970 is biased outward by a biasing force of first spring 976 such that output gear 969 is aligned with internal teeth 972, and emergency button 990 is biased outward such that plate portion 992 is disposed in sidewall 912 of base 910. In some embodiments, sidewall 912 includes a cavity through which can be reached. In some embodiments, in the operational state, plate portion 992 is located inside the cavity such that its surface is flush with the surface of sidewall 912.

However, in the case of an emergency such as the above-described malfunctions of actuator 900 or damage to actuator 900, while cuff 810 is contracted around urethra 801, emergency button 990 may be manipulated to loosen cables 822 and thereby release cuff 810 such that cuff 810 does not prevent flow of urine through urethra 801. In particular, emergency button 990 may function as a release mechanism for disengaging the actuator from the cuff in the manner detailed below.

For example, as shown in FIG. 9L, emergency button 990 may be pressed inward in the direction of arrows P to disengage output shaft 968 from first pulley 970. More specifically, when plate portion 992 of emergency button 990 is pressed inward as illustrated in FIG. 9L (e.g., by a user's finger or thumb, or by an object held by the user), first pulley 970 is pressed inward by projecting portion 994 of emergency button 990, against the biasing force of first spring 976. First pulley 970 may thus move to the extent that output gear 969 disengages from internal teeth 972 and, therefore, output shaft 968 disengages from first pulley 970. When output shaft 968 is disengaged from first pulley 970 while cuff 810 is in the contracted state, first pulley 970 is able to rotate in counterclockwise unwinding direction R4 without resistance from gear assembly 960, motor 944, and magnet assembly 950, which allows cables 822 to loosen, cuff 810 to expand, and urethra 801 to open.

When plate portion 992 of emergency button 990 is released after being pressed inward, the biasing force of first spring 976 pushes first pulley 970 outward and into alignment with internal teeth 972, and emergency button 990 is thereby moved outward by first pulley 970 pushing against projecting portion 994 such that plate portion 992 of emergency button 990 is disposed in sidewall 912 of base 910. Thus, by releasing emergency button 990, actuator 900 is returned to a normal operational state.

Some safety mechanisms may address situations in which cables 822 become bound or tangled inside actuator 900. One such safety mechanism may include an emergency access hole 928 disposed in sidewall 912 of base 910, shown in FIG. 9C. Emergency access hole 928 may allow a user to insert a thin cutting tool to cut cables 822 in an emergency. Emergency access hole 928 may be covered by outer shell 902 during normal use of actuator 900 to ensure the interior of housing 904 is sealed from external fluids. Thus, in such embodiments, outer shell 902 would need to be punctured by the cutting tool to access emergency access hole 928 and cables 822.

FIG. 9M schematically illustrates an example of an implementation in which actuator 900 is operated by an electronic device ED. Electronic device ED may be an electronic device configured to emit an electromagnetic field F1 for inductive coupling. In some embodiments, electronic device ED is a mobile telephone or other mobile electronic device. In some embodiments, electronic device ED is configured to perform inductive coupling according to near-field communication (NFC) standards.

Some embodiments include mechanisms for operating or controlling the artificial sphincter using electromagnetic signals generated outside the body. In some embodiments the electromagnetic signals may be generated by an electronic device, or a permanent magnet. In an embodiment shown in FIG. 9M, for example, electronic device ED, positioned within a specified distance of wire coil 948, emits electromagnetic field F1. The specified distance may be within the induction range, the range in which electromagnetic field F1 can induce an electric current I in wire coil 948. Wire coil 948 transfers the induced current I to electronic board 940. For example, referring to FIGS. 9B and 9E, wire coil 948 may transfer induced current I to rectifier 941, and rectifier 941 may output a rectified current corresponding to induced current I to supply power to electronic board 940 and motor 944. Controller 942 may control motor driver 943 to cause motor driver 943 to operate motor 944 based on a data signal S provided from electronic device ED through electromagnetic field F1.

In some embodiments controller 942 includes a chipset storing a variety of information. The information may include commands for adjusting the output voltage at different levels. Further, controller 942 may include a central timer utilized for transition of the sphincter from one state to another state based on the duration of the magnetic induction. For example, if the magnetic induction lasts for a predefined duration (such as 5 seconds, 10 seconds, etc.) the sphincter may switch from one of the closed (contracted) and open (released) states to the other one. That is, if the sphincter is in the closed state (the cuff being contracted) it may transition to the open state by releasing the cuff. Conversely, if the sphincter is in the open state (the cuff being released) it may transition to the closed state by contracting the cuff. In another example, if the induction lasts for a longer time (e.g., 1 minute, 2 minutes, etc.), the sphincter may enter a setting phase in which it allows the user to change one or more of the settings using durations or changes in the induction or using a settings screen on, for example, an app on the user's smart phone or smart watch.

In various embodiments, the data signal S may depend on one or more characteristics of the electromagnetic field F1, such as its duration, its frequency, its magnitude, etc. In some embodiments, electronic device ED may include an electromagnetic field generator for which one or more of those characteristics can be controlled. Moreover, in some embodiments, electronic device ED may include an interface through which a user may be able to control those characteristics. For example, electronic device ED may be a smartphone that includes hardware for generating the electromagnetic field and a software, such as an app, for controlling those characteristics of the electromagnetic field. Alternatively, the electronic device may be a mobile phone that generates an electromagnetic field with a constant frequency or magnitude. In some embodiments, a user may start and stop the induction of the induced current by moving a mobile phone in and out of the induction range. For example, the user may bring the mobile phone near the location of the wire coil to start induction and move it away from that location to stop the induction. Accordingly, the user may control the duration of the induction by holding the mobile phone near the wire coil for the desired duration before moving it away.

Controller 942 may utilize the data signal to control the operation of the artificial sphincter. As an example, in some embodiments, controller 942 may control motor driver 943 to rotate motor 944 in the clockwise direction R1 to contract cuff 810 or rotate motor 944 in the counterclockwise direction R2 to expand cuff 810 depending on a duration (length of time) electromagnetic field F1 is applied to wire coil 948.

For example, in some embodiments, controller 942 may control motor driver 943 to rotate motor 944 in the clockwise direction R1 to tighten cuff 810 in response to electromagnetic field F1 being applied to wire coil 948 for a duration that is greater than a first threshold duration, and may control motor driver 943 to rotate motor 944 in the counterclockwise direction R2 to loosen cuff 810 in response to electromagnetic field F1 being applied to wire coil 948 for a duration that is less than the first threshold duration and greater than a second threshold duration. As a non-limiting example, the first threshold duration may be 5 seconds and the second threshold duration may be 2 seconds. In other embodiments, controller 942 may control motor driver 943 to rotate motor 944 in the counterclockwise direction R2 to loosen cuff 810 in response to electromagnetic field F1 being applied to wire coil 948 for a duration that is greater than the first threshold duration, and may control motor driver 943 to rotate motor 944 in the clockwise direction R1 to tighten cuff 810 in response to electromagnetic field F1 being applied to wire coil 948 for a second duration that is less than the first threshold duration and greater than the second threshold duration.

In some embodiments, controller 942 may control motor driver 943 not only based on the data signal (e.g., a duration of the induction) but further based on the cuff state information. For example, controller 942 may control motor driver 943 to rotate motor 944 in the clockwise direction R1 to tighten cuff 810 in response to electromagnetic field F1 being applied to wire coil 948 for a duration that is greater than a threshold duration and the cuff state information indicating that cuff 810 is currently in the loosened state. On the other hand, controller 942 may control motor driver 943 to rotate motor 944 in the counterclockwise direction R2 to loosen cuff 810 in response to electromagnetic field F1 being applied to wire coil 948 for the duration that is greater than the threshold duration and the cuff state information indicating that cuff 810 is currently in the tightened state.

Additionally, electronic device ED may be used to configure the degree to which cuff 810 contracts and expands. For example, electronic device ED may provide data to controller 942 through electromagnetic field F1 to set a minimum inner diameter of cuff 810 when cuff 810 is fully contracted and a maximum inner diameter of cuff 810 when cuff is fully expanded. For example, electronic device ED may set the minimum and maximum inner diameters of cuff 810 by setting a number of revolutions performed by motor 944 or a duration motor 944 operates when contracting and expanding cuff 810. Thus, controller 942 may precisely control contraction and expansion sizes of cuff 810 such that the sphincter can operate effectively and safely when used with urethras of various sizes, for example, urethras with diameters between 5-50 mm.

Various embodiments may use one or more types of magnets for operating the sphincter in different manners. FIG. 9N schematically illustrates an example of such an embodiment in which the actuator is operated by a magnet M. In some embodiments, magnet M is a permanent magnet. Magnet M may be used to contract and expand cuff 810 as an alternative to using motor 944, or in the event that motor 944 fails to operate. Magnet M may produce magnetic fields that cause magnet assembly 950 to rotate when magnet M is positioned within a specified distance of magnet assembly 950. For example, as shown in FIG. 9N, magnet M may have a first pole that produces a first magnetic field F2 and a second pole that produces a second magnetic field F3. When magnet M is positioned within a specified distance of magnet assembly 950 such that the first pole of magnet M faces magnet assembly 950, first electromagnetic field F2 may interact with magnetic fields produced by first and second magnets 952 and 954 of magnet assembly 950, thereby causing first and second magnets 952 and 954, magnet support 951, and magnet assembly output shaft 956 to rotate together in a clockwise direction R5 to contract cuff 810. On the other hand, when magnet M is positioned within a specified distance of magnet assembly 950 such that the second pole of magnet M faces magnet assembly 950, second electromagnetic field F3 may interact with the magnetic fields produced by first and second magnets 952 and 954 of magnet assembly 950, thereby causing first and second magnets 952 and 954, magnet support 951, and magnet assembly output shaft 956 to rotate together in a counterclockwise direction R6 to expand cuff 810.

In some embodiments, one or more of the alternative mechanisms may be positioned differently compared to those explained above. As an example, FIG. 11 shows a complex 1100 in which the magnet assembly is positioned in line with the motor shaft. Complex 1100 includes a motor 1144, a motor shaft 1146, a magnet assembly 1150, a gear assembly 1160, and a first pulley 1170. As shown, in complex 1100, magnet assembly 1150 is mounted on motor shaft 1146 and is therefore connected in-line with motor 1144, instead of being connected to gear assembly 1160 by a separate shaft.

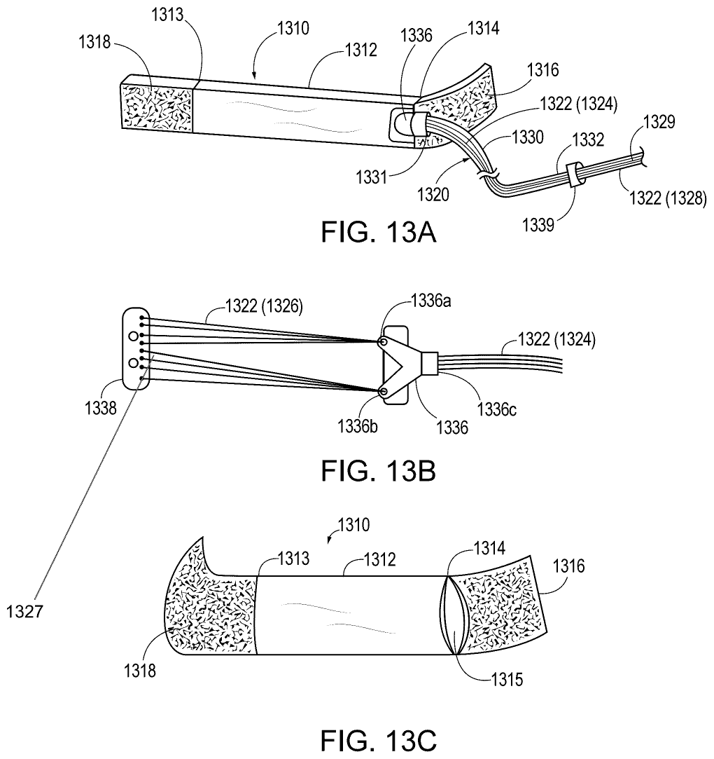

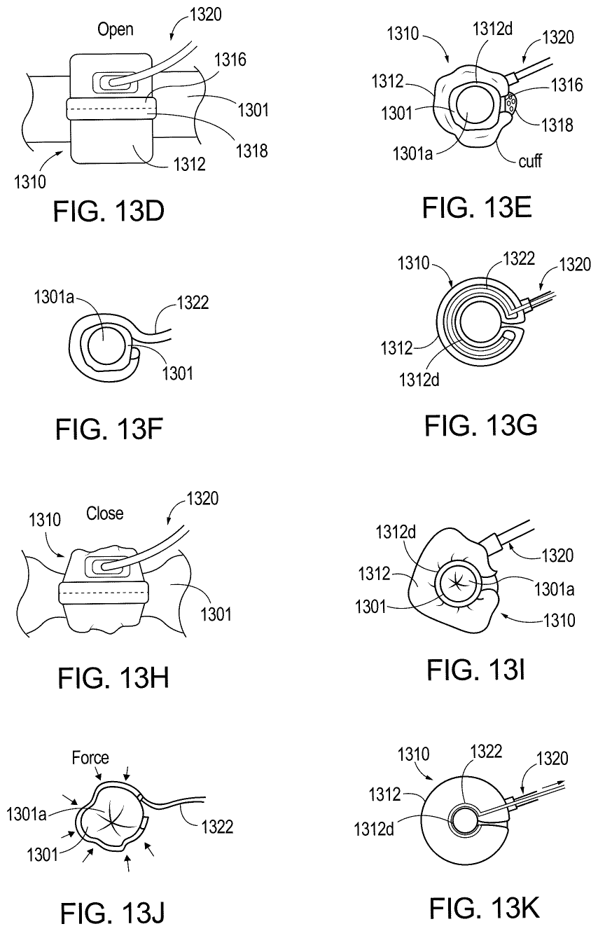

Various embodiments may utilize different designs for the cuff or the connection part. FIGS. 13A-13C show some details of a cuff 1310 and a connection part 1320 according to some embodiments. Cuff 1310 includes a cuff body 1312, an internal pocket 1315 disposed in cuff body 1312, a first attaching member 1316, and a second attaching member 1318.

Cuff body 1312 itself has a proximal end 1313 and a distal end 1314. In some embodiments, cuff body 1312 may be constructed of multiple layers. For example, cuff body 1312 may include inner, middle, and outside layers similar to inner, middle, and outside layers 812a, 812b, and 812c of cuff body 812 shown in FIG. 8A.

Connection part 1320 includes cables 1322, a cable jacket 1330 having a first end 1331 adjacent to cuff 1310 and a second end 1332 that may be positioned adjacent to an actuator, a proximal cable support 1336, a distal cable support 1338, and a connector 1339 attached to second end 1332 of cable jacket 1330.

In some embodiments, eight or more cables 1322 may be provided. However, fewer or more than eight cables 1322 may be provided in other embodiments. In some embodiments, cables 1322 may each be a filament or a spun fiber. For example, cables 1322 may be nylon monofilaments, while other materials and constructions are also possible. Middle segments 1324 of cables 1322 extend inside cable jacket 1330 between cuff 1310 and an actuator. First end segments 1326 of cables 1322 are disposed in internal pocket 1315 of cuff 810. First end segments 1326 extend through openings in proximal cable support 1336 and include first ends 1327 of cables 1322 that are fixed to distal cable support 1338. For example, first and second groups of cables 1322 may be respectively routed through a first and second entry openings 1336a and 1336b of proximal cable support 1336, and may converge at exit opening 1336c of proximal cable support 1336.

Proximal cable support 1336 is disposed inside cuff body 1312, adjacent a first end 1331 of cable jacket 1330, and is attached to proximal end 1313 of cuff body 1312. Distal cable support 1338 is disposed inside cuff body 1312 and is attached to distal end 1314 of cuff body 1312.

Connector 1339 of connection part 1320 may be connected to the actuator device. For example, connector 1339 may be a threaded connector configured to connect to a threaded portion of the actuator device.

Second end segments 1328 of cables 1322 extend from second end 1332 of cable jacket 1330 and terminate at second ends 1329 of cables 1322. Second end segments 1328 may be disposed inside the actuator, where the second end segments 1328 may engage cuff actuating components of the actuator device that are operable to control (e.g., pull and release) cables 1322 to selectively deploy (e.g., contract or buckle) or release (e.g., relax) cuff 1310.

In various embodiments, the combination of the cuff and the connection part inside the cuff may provide a sheathed belt mechanism (hereinafter also called the belt for brevity) to control the flow of the bodily liquid inside the lumen safely and comfortably. More specifically, in this mechanism, the actuator may tighten the connection part inside the belt and thus, for example, reduce the length of the connection part inside the belt. This tightening, in turn, may cause the belt to tighten around the lumen, thus reducing, or fully blocking, the flow of the bodily liquid in the lumen. This configuration of the belt may be termed a deployed, buckled, or tightened state.

Conversely, the actuator may loosen the connection part inside the belt, causing the belt to loosen around the lumen, and allowing the lumen to expand (i.e., increase its diameter under the belt), in turn increasing the length of the connection part inside the belt. Such loosening of the belt and expansion of the lumen may allow the bodily fluid to flow more freely through the lumen. This configuration of the belt may be termed an undeployed, relaxed, or loosened state.

In various embodiments, thus, the connection part may provide the internal mechanism of the belt for the tightening or loosening of the belt. The cuff, on the other hand, may provide the intervening layer between the connection part and the lumen (or any body part around which the belt is wrapped, such as the limb described above). The cuff, therefore, may reduce the risk of injury to the lumen or discomfort to the patient, which may arise if the connection part is in direct contact with the lumen. In some embodiments, the cuff is designed such that it can change its form between the undeployed and deployed states, thus accommodating the changes in the length of the connection part inside the belt.

FIGS. 13D-13K illustrate various features of cuff 1310 enabling the cuff to tighten or loosen around a body lumen according to various embodiments. More specifically, cuff 1310 may be configured to encircle a lumen 1301 (such as a urethra) of a patient.

When cuff 1310 encircles lumen 1301, cuff 1310 may form a closed loop around lumen 1301, such that proximal end 1313 and distal end 1314 of cuff body 1312 are secured together or are adjacent to each other. Moreover, in this configuration, first and second attaching members 1316 and 1318 may be attached to each other. For example, in some embodiments, first and second attaching members 1316 and 1318 may be sutured, glued, or otherwise fastened to each other to secure cuff 1310 in the form of a closed loop. In some embodiments, first and second attaching members 1316 and 1318 may be made of a mesh material that facilitates attachment of first and second attaching members 1316 and 1318 to each other by, for example, stitching.

FIGS. 13D-13G illustrate cuff 1310 and connection part 1320 when cuff 1310 is in an undeployed state. More specifically, FIGS. 13D and 13E show cuff 1310 and connection part 1320 with cuff body 1312 and cables 1322 wrapped around lumen 1301 in the undeployed state. FIG. 13F shows cables 1322 in the undeployed state, with cuff 1310 removed for illustrative purposes. FIG. 13G shows cuff 1310 and connection part 1320 with cuff body 1312 and cables 1322 wrapped in a closed loop in the undeployed state, with lumen 1301 not shown for illustrative purposes. In the undeployed state, cables 1322 are not pulled by the actuator device or have been released by the actuator device. Thus, cables 1322 are loosely wrapped around lumen 1301 inside cuff body 1312, as shown in FIG. 13F. As a result, cables 1322 do not apply significant squeezing force on cuff body 1312 and cuff body 1312 is loosely wrapped around lumen 1301, thereby allowing lumen duct 1301a to open or maintaining lumen duct 1301a in an open state such that bodily liquid can pass therethrough, as shown in FIGS. 13D and 13E.

FIGS. 13H-13K, on the other hand, illustrate cuff 1310 and connection part 1320 when cuff 1310 is in a deployed state. More specifically, FIGS. 13H and 13I show cuff 1310 and connection part 1320 with cuff body 1312 and cables 1322 wrapped around lumen 1301 in the deployed state. FIG. 13J shows cables 1322 in the deployed state, with cuff 1310 removed for illustrative purposes. FIG. 13K shows cuff 1310 and connection part 1320 with cuff body 1312 and cables 1322 wrapped in a closed loop in the deployed state, with lumen 1301 not shown for illustrative purposes. In the deployed state, cables 1322 are pulled by the actuator device. Thus, cables 1322 are tightened inside cuff body 1312 itself around lumen 1301, as shown in FIG. 13J. When tightened, cables 1322 apply a squeezing force on an inner wall 1312d of cuff body 1312. As a result, inner wall 1312d of cuff body 1312 is deformed/buckled, thereby reducing the inner diameter of cuff 1310 such that cuff body 1312 is tightly wrapped around lumen 1301 to close lumen duct 1301a, in turn reducing or blocking flow of the bodily liquid inside lumen 1301, as illustrated in FIGS. 13H and 13I.

Some embodiments provide a controllable cover for contracting or releasing some extended parts of the body such as the bladder or stomach. The controllable cover may include a compressing mesh, and layer, a band, or similar types of surfaces. The controllable cover may enable a user to controllably contract the body part or allow the body part to expand. In various embodiments, the controllable cover may include a cuff, a connection part, and an actuator similar to those of the embodiments described above in relation to the artificial sphincter. In some embodiments, however, the cuff or the connection part of the controllable cover may be designed eventually from the above-discussed cuff or connection part. In particular, the cuff may be designed as an extended cover to wrap around at least part of the body part, as further detailed below.

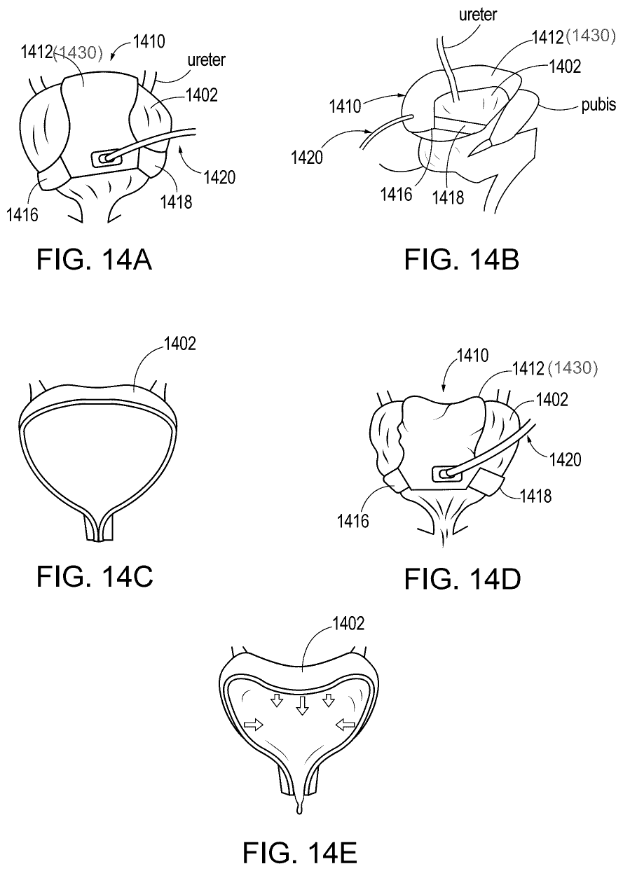

FIGS. 14A-14C show some details of a controllable cover including a cuff 1410 and a connection part 1420 according to some embodiments. As generally stated above for similarly named parts, cuff 1410 may be similar to one or more of cuffs 710, 810, and 1310, and connection part 1420 may be similar to one or more of connection parts 720, 820, and 1320. However, whereas cuffs 710, 810, and 1310, and connection parts 720, 820, and 1320 are shaped and sized for use with a lumen, cuff 1410 and connection part 1420 are shaped and sized for use with a body parts such as a bladder 1402. More specifically, cuff 1410 and connection part 1420 may be configured to enable a patient who lacks neurological/muscular control of bladder 1402 to control discharge of urine from bladder 1402.

Cuff 1410 includes a cuff body 1412, a first attaching member 1416, and a second attaching member 1418. Similar to connection part 1320 of FIGS. 13A-13K, connection part 1420 may include a cable jacket 1430, cables (not shown) extending in cable jacket 1430 and an internal pocket (not shown) of cuff body 1412, a proximal cable support (not shown), and a distal cable support (not shown).

Cuff 1410 may be configured to encircle bladder 1402, as shown in FIGS. 14A, 14B, and 14D. In various embodiments, encircling a body parts such as the bladder may indicate covering the body part partially or in full, such that the cuff is configured to perform the functions described in this disclosure. In some embodiments, when cuff 1410 encircles a bladder 1402, cuff 1410 may form a closed or semi-closed layer or band around bladder 1402

First and second attaching members 1416 and 1418 may be attached to each other when cuff 1410 is configured in the closed loop and placed around bladder 1402. For example, in some embodiments, first and second attaching members 1416 and 1418 may be sutured, glued, or otherwise fastened to each other to secure cuff 1410 in the form of a closed loop.

FIGS. 14A-14C illustrate cuff 1410, connection part 1420, and bladder 1402 when cuff 1410 is in an undeployed or relaxed state. More specifically, FIGS. 14A and 14B show cuff 1410 and connection part 1420 with cuff body 1412 and cables wrapped around bladder 1402 in the undeployed state. FIG. 14C shows bladder 1402 in an expanded state corresponding to the undeployed state of cuff 1410. In the undeployed state, cables of connection part 1420 are not pulled by the actuator device or have been released by the actuator device. As a result, the cables do not apply significant squeezing force on cuff body 1412 and cuff body 1412 is loosely wrapped around bladder 1402. Therefore, bladder 1402 is allowed to remain expanded.

FIGS. 14D and 14E, on the other hand, illustrate cuff 1410, connection part 1420, and bladder 1402 when cuff 1410 is in a deployed or buckled state. More specifically, FIG. 14D shows cuff 1410 and connection part 1420 with cuff body 1412 and the cables wrapped around bladder 1402 in the deployed state. FIG. 14E shows bladder 1402 in a contracted or compressed state corresponding to the deployed state of cuff 1410. In the deployed state, the cables may be pulled by the actuator device. Thus, the cables are tightened around bladder 1402 inside cuff body 1412. When tightened around bladder 1402, the cables apply a squeezing force on an inner wall of cuff body 1412. As a result, the inner wall of cuff body 1412 is deformed/buckled, thereby reducing the inner diameter of cuff 1410 such that cuff body 1412 contracts bladder 1402 causing the urine to flow out of bladder 1402.

[? We may need to add some closing statements summarizing the disclosure, but that may not be necessary. We can decide later]

In various embodiments, one or more of disclosed modules may be implemented via one or more computer programs for performing the functionality of the corresponding modules, or via computer processors executing those programs. In some embodiments, one or more of the disclosed modules may be implemented via one or more hardware units executing firmware for performing the functionality of the corresponding modules. In various embodiments, one or more of the disclosed modules may include storage media for storing data used by the module, or software or firmware programs executed by the module. In various embodiments, one or more of the disclosed modules or disclosed storage media may be internal or external to the disclosed systems. In some embodiments, one or more of the disclosed modules or storage media may be implemented via a computing “cloud”, to which the disclosed system connects via a network connection and accordingly uses the external module or storage medium. In some embodiments, the disclosed storage media for storing information may include non-transitory computer-readable media, such as a CD-ROM, a computer storage, e.g., a hard disk, or a flash memory. Further, in various embodiments, one or more of the storage media may be non-transitory computer-readable media that store data or computer programs executed by various modules, or implement various techniques or flow charts disclosed herein.

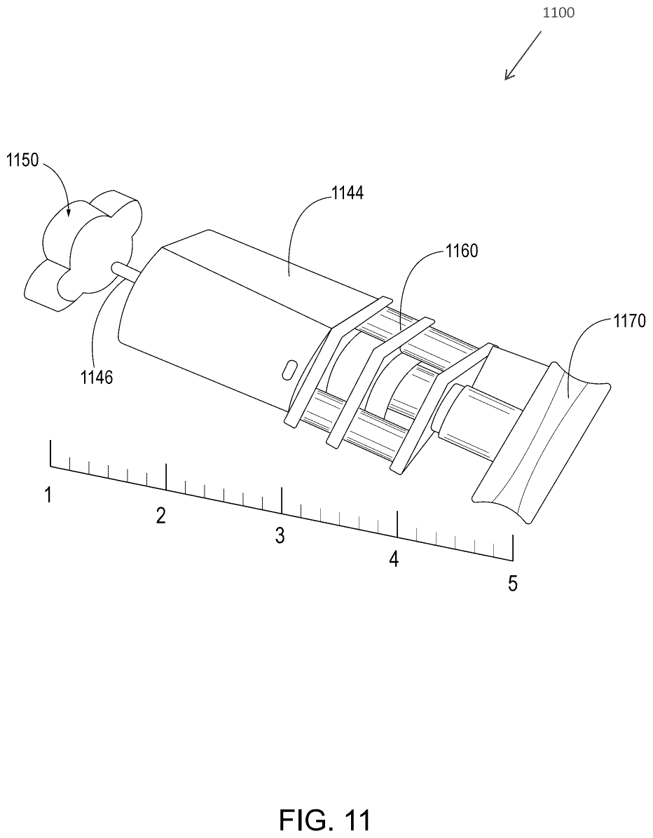

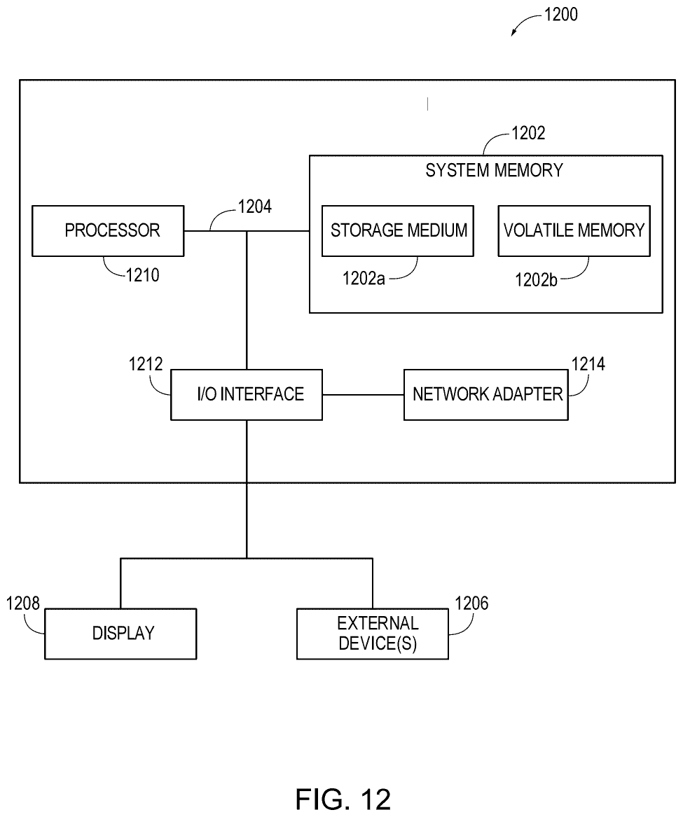

By way of example, FIG. 12 schematically depicts an example of an implementation of a controller 1200 according to some embodiments. Controller 1200 may correspond to controller 942 in the embodiment of FIG. 9B and may therefore be configured to perform similar functions. Controller 1200 includes a system memory 1202 that may include a permanent memory module (e.g., ROM 1202a) and a transient memory module (e.g., RAM 1202b), an internal bus xx04, a processor 1210 (e.g., a microprocessor), an I/O interface 1212, and a communication interface 1214 (such as a network adapter). I/O interface 1212 may be in communication with one or more external input devices 1206 (such as a mouse, a keyboard, or a touch screen) or output devices 1208 (such as a display, a printer, or a speaker).

Processor 1210 and the system memory 1202 may be utilized to store and execute instructions performing the function of controller 1200. Moreover, internal bus 1204 may enable communication between the processor and other parts of controller 1200 such as system memory 1202, I/O interface 1212, or communication interface 1214.

Some or all of the method steps may be executed by (or using) a hardware apparatus, like for example, a processor, a microprocessor, a programmable computer or an electronic circuit. In some embodiments, some one or more of the most important method steps may be executed by such an apparatus.

Depending on certain implementation requirements, embodiments of the disclosure may be implemented in hardware and/or in software. The implementation may be performed using a non-transitory storage medium such as a digital storage medium, for example a floppy disc, a DVD, a Blu-Ray, a CD, a ROM, a PROM, and EPROM, an EEPROM or a FLASH memory, having electronically readable control signals stored thereon, which cooperate (or are capable of cooperating) with a programmable computer system such that the respective method is performed. Therefore, the digital storage medium may be computer readable.

Those having ordinary skill will appreciate that various changes may be made to the above embodiments without departing from the scope of the disclosure.

Although some aspects have been described in the context of a system or an apparatus, it is clear that these aspects may also represent a description of the corresponding method, where a block or device corresponds to a method step or a feature of a method step. Analogously, aspects described in the context of a method step also represent a description of a corresponding block or item or feature of a corresponding apparatus.

The foregoing description of the embodiments has been presented for purposes of illustration only. It is not exhaustive and does not limit the embodiments to the precise form disclosed. While several exemplary embodiments and features are described, modifications, adaptations, and other implementations may be possible, without departing from the spirit and scope of the embodiments. Accordingly, unless explicitly stated otherwise, the descriptions relate to one or more embodiments and should not be construed to limit the embodiments as a whole. This is true regardless of whether or not the disclosure states that a feature is related to “a,” “the,” “one,” “one or more,” “some,” or “various” embodiments. As used herein, the singular forms “a,” “an,” and “the” may include the plural forms unless the context clearly dictates otherwise. Further, the term “coupled” does not exclude the presence of intermediate elements between the coupled items. Also, stating that a feature may exist indicates that the feature may exist in one or more embodiments.

In this disclosure, the terms “include,” “comprise,” “contain,” and “have,” when used after a set or a system, mean an open inclusion and do not exclude addition of other, non-enumerated, members to the set or to the system. Further, unless stated otherwise or deducted otherwise from the context, the conjunction “or,” if used, is not exclusive, but is instead inclusive to mean and/or.

Moreover, if these terms are used, a set may include one or more members, and a subset of a set may include one or more than one, including all, members of the set.

Further, if used in this disclosure, and unless stated or deducted otherwise, a first variable is an increasing function of a second variable if the first variable does not decrease and instead generally increases when the second variable increases. On the other hand, a first variable is a decreasing function of a second variable if the first variable does not increase and instead generally decreases when the second variable increases. In some embodiment, a first variable may be an increasing or a decreasing function of a second variable if, respectively, the first variable is directly or inversely proportional to the second variable.

The disclosed compositions, systems, methods, and apparatus are not limited to any specific aspect or feature or combinations thereof, nor do the disclosed compositions, systems, methods, and apparatus require that any one or more specific advantages be present or problems be solved. Any theories of operation are to facilitate explanation, but the disclosed compositions, systems, methods, and apparatus are not limited to such theories of operation.

Modifications and variations are possible in light of the above teachings or may be acquired from practicing the embodiments. For example, the described steps need not be performed in the same sequence discussed or with the same degree of separation. Likewise various steps may be omitted, repeated, combined, or performed in parallel, as necessary, to achieve the same or similar objectives. Similarly, the systems described need not necessarily include all parts described in the embodiments, and may also include other parts not described in the embodiments. Accordingly, the embodiments are not limited to the above-described details, but instead are defined by the appended claims in light of their full scope of equivalents. Further, the present disclosure is directed toward all novel and non-obvious features and aspects of the various disclosed embodiments, alone and in various combinations and sub-combinations with one another.

While the present disclosure has been particularly described in conjunction with specific embodiments, many alternatives, modifications, and variations will be apparent in light of the foregoing description. It is therefore contemplated that the appended claims will embrace any such alternatives, modifications, and variations as falling within the true spirit and scope of the present disclosure.

What is claimed is:

1. An implantable device for adjusting fluid flow through a bodily lumen, the implantable device comprising:
a cuff configured to be positioned around the lumen;
an actuator;
a connection part connecting the cuff and the actuator; and
an induction coil configured to generate an induced current in response to a transcutaneous magnetic induction,
wherein:
the actuator is configured to operate the cuff by transitioning the cuff between a deployed state and an undeployed state in response to the induced current.

2. The device of claim 1, further comprising an alternative actuation mechanism that includes a magnet assembly configured to rotate and cause the transition of the cuff between the deployed state and the undeployed state.

3. The device of claim 1, wherein the lumen is a urethra, an artery, a vein, or a colon.

4. The device of claim 1, wherein the actuator includes a motor configured to be powered by the induced current.

5. The device of claim 4, wherein the motor is configured to be powered by a rectified current generated from the induced current.

6. The device of claim 1, wherein the actuator is configured to operate the cuff without using a battery.

7. The device of claim 1, wherein the actuator is configured to operate the cuff based on information derived from the induced current.

8. The device of claim 1, wherein the transcutaneous magnetic induction is generated by an external inducer.

9. The device of claim 8, wherein the external inducer is configured to induce a temporally varying magnetic flux in the coil to generate the induced current.

10. The device of claim 8, wherein the external inducer is configured such that a movement of the external inducer relative to the induction coils generates a varying magnetic flux in the induction coil and generating the induced current.

11. An implantable device for adjusting fluid flow through a bodily lumen, the implantable device comprising:
a cuff configured to be positioned around the lumen;
an actuator;
a connection part connecting the cuff and the actuator; and
a fail-safe mechanism,
wherein:
the actuator is configured to operate the cuff by transitioning the cuff between a deployed state and an undeployed state; and
the fail-safe mechanism is configured to transition the cuff from the deployed state to the undeployed state.

12. The device of claim 11, wherein the fail-safe mechanism includes a magnet assembly configured to rotate and cause the transition of the cuff from the deployed state to the undeployed state.

13. The device of claim 11, wherein the fail-safe mechanism includes a release mechanism configured to disengage the actuator from the cuff.

14. The device of claim 11, wherein the fail-safe mechanism is configured to be operated when the actuator fails to operate the cuff by transitioning the cuff from the deployed state to the undeployed state.

15. An implantable device for controllably covering a body part, the device comprising:
an actuator; and
a belt including:
a cuff configured to wrap around at least part of the body part; and
a connection part connecting the cuff and the actuator,
wherein:
the cuff has two ends configured to be fixedly connected to position the cuff around the body part;
the actuator is configured to operate the belt by transitioning the belt between a deployed state and an undeployed state; and
the cuff provides an intervening sheath between the connection part and the body part.

16. The device of claim 15, wherein the cuff includes a mesh.

17. The device of claim 15, wherein in the deployed state the cuff buckles around the body part to increase a pressure on the body part.
